SEQUENCE LISTING (30 sequences)

SEQ ID NO: 1 - RNA, 37 nt, synthetic construct
  source 1..37: mol_type = other RNA
agtgtgtgtgtgtgtgtgtgtgtgtgtgtgtgtgtgt

SEQ ID NO: 2 - RNA, 33 nt, synthetic construct
  source 1..33: mol_type = other RNA
gagagagcgcgtcagagacttggtggtgcataa

SEQ ID NO: 3 - RNA, 36 nt, synthetic construct
  source 1..36: mol_type = other RNA
gtgtgaatgaatgtgtgaatgaatgtgtgaatgaat

SEQ ID NO: 4 - RNA, 13 nt, synthetic construct
  source 1..13: mol_type = other RNA
agtgtgtgtgtgt

SEQ ID NO: 5 - DNA, 13 bp, synthetic construct
  source 1..13: mol_type = other DNA
agtgtgtgtgtgt

SEQ ID NO: 6 - RNA, 13 nt, synthetic construct
  source 1..13: mol_type = other RNA
  modified_base 2: mod_base = gm
  modified_base 3: mod_base = um
  modified_base 4: mod_base = gm
  modified_base 5: mod_base = um
  modified_base 6: mod_base = gm
  modified_base 7: mod_base = um
  modified_base 8: mod_base = gm
  modified_base 9: mod_base = um
  modified_base 10: mod_base = gm
  modified_base 11: mod_base = um
  modified_base 12: mod_base = gm
  modified_base 13: mod_base = um
  misc_feature 1..13: note = phosphorothioate bond
  modified_base 1: mod_base = OTHER; note = 2'-O-Methyladenosine
agtgtgtgtgtgt

SEQ ID NO: 7 - RNA, 34 nt, synthetic construct
  source 1..34: mol_type = other RNA
  modified_base 1: mod_base = gm
  modified_base 2: mod_base = OTHER; note = 2'-O-Methyladenosine
  modified_base 3: mod_base = gm
  modified_base 4: mod_base = OTHER; note = 2'-O-Methyladenosine
  modified_base 5: mod_base = gm
  modified_base 6: mod_base = OTHER; note = 2'-O-Methyladenosine
  modified_base 7: mod_base = gm
  modified_base 8: mod_base = cm
  modified_base 9: mod_base = gm
  modified_base 10: mod_base = cm
  modified_base 11: mod_base = gm
  modified_base 12: mod_base = um
  modified_base 13: mod_base = gm
  modified_base 14: mod_base = cm
  modified_base 15: mod_base = OTHER; note = 2'-O-Methyladenosine
  modified_base 16: mod_base = gm
  modified_base 17: mod_base = OTHER; note = 2'-O-Methyladenosine
  modified_base 18: mod_base = gm
  modified_base 19: mod_base = OTHER; note = 2'-O-Methyladenosine
  modified_base 20: mod_base = cm
  modified_base 21: mod_base = um
  modified_base 22: mod_base = um
  modified_base 23: mod_base = gm
  modified_base 24: mod_base = gm
  modified_base 25: mod_base = um
  modified_base 26: mod_base = gm
  modified_base 27: mod_base = gm
  modified_base 28: mod_base = um
  modified_base 29: mod_base = gm
  modified_base 30: mod_base = cm
  modified_base 31: mod_base = OTHER; note = 2'-O-Methyladenosine
  modified_base 32: mod_base = um
  modified_base 33: mod_base = OTHER; note = 2'-O-Methyladenosine
  modified_base 34: mod_base = OTHER; note = 2'-O-Methyladenosine
  misc_feature 1..34: note = phosphorothioate bond
gagagagcgcgtgcagagacttggtggtgcataa

SEQ ID NO: 8 - DNA, 13 bp, synthetic construct
  source 1..13: mol_type = other DNA
  misc_feature 1^2: note = phosphorothioate bond
  misc_feature 3^4: note = phosphorothioate bond
  misc_feature 7^8: note = phosphorothioate bond
  misc_feature 8^9: note = phosphorothioate bond
  misc_feature 10^11: note = phosphorothioate bond
  misc_feature 12^13: note = phosphorothioate bond
  misc_feature 6^7: note = phosphorothioate bond
  modified_base 1: mod_base = OTHER; note = 2'-O-methoxy-ethyl-adenosine
  modified_base 2: mod_base = OTHER; note = 2'-O-methoxy-ethyl-guanosine
  modified_base 3: mod_base = OTHER; note = 2'-O-methoxy-ethyl-thymidine
  modified_base 4: mod_base = OTHER; note = 2'-O-methoxy-ethyl-guanosine
  modified_base 5: mod_base = OTHER; note = 2'-O-methoxy-ethyl-thymidine
  modified_base 6: mod_base = OTHER; note = 2'-O-methoxy-ethyl-guanosine
  modified_base 7: mod_base = OTHER; note = 2'-O-methoxy-ethyl-thymidine
  modified_base 8: mod_base = OTHER; note = 2'-O-methoxy-ethyl-guanosine
  modified_base 9: mod_base = OTHER; note = 2'-O-methoxy-ethyl-thymidine
  modified_base 10: mod_base = OTHER; note = 2'-O-methoxy-ethyl-guanosine
  modified_base 11: mod_base = OTHER; note = 2'-O-methoxy-ethyl-thymidine
  modified_base 12: mod_base = OTHER; note = 2'-O-methoxy-ethyl-guanosine
  modified_base 13: mod_base = OTHER; note = 2'-O-methoxy-ethyl-thymidine
  misc_feature 5^6: note = phosphorothioate bond
agtgtgtgtgtgt

SEQ ID NO: 9 - RNA, 13 nt, synthetic construct
  source 1..13: mol_type = other RNA
  misc_feature 1^2: note = phosphorothioate bond
  misc_feature 3^4: note = phosphorothioate bond
  misc_feature 5^6: note = phosphorothioate bond
  misc_feature 6^7: note = phosphorothioate bond
  misc_feature 7^8: note = phosphorothioate bond
  misc_feature 8^9: note = phosphorothioate bond
  misc_feature 10^11: note = phosphorothioate bond
  misc_feature 12^13: note = phosphorothioate bond
  modified_base 1: mod_base = OTHER; note = 2'-O-methoxy-ethyl-adenosine
  modified_base 2: mod_base = OTHER; note = 2'-O-methoxy-ethyl-guanosine
  modified_base 3: mod_base = um
  modified_base 4: mod_base = OTHER; note = 2'-O-methoxy-ethyl-guanosine
  modified_base 5: mod_base = um
  modified_base 6: mod_base = OTHER; note = 2'-O-methoxy-ethyl-guanosine
  modified_base 7: mod_base = um
  modified_base 8: mod_base = OTHER; note = 2'-O-methoxy-ethyl-guanosine
  modified_base 9: mod_base = um
  modified_base 10: mod_base = OTHER; note = 2'-O-methoxy-ethyl-guanosine
  modified_base 11: mod_base = um
  modified_base 12: mod_base = OTHER; note = 2'-O-methoxy-ethyl-guanosine
  modified_base 13: mod_base = um
agtgtgtgtgtgt

SEQ ID NO: 10 - RNA, 13 nt, synthetic construct
  source 1..13: mol_type = other RNA
acacacacacaca

SEQ ID NO: 11 - RNA, 32 nt, synthetic construct
  source 1..32: mol_type = other RNA
cgcgacggacggaaagacccctatccgtcgcg

SEQ ID NO: 12 - DNA, 37 bp, synthetic construct
  source 1..37: mol_type = other DNA
agtgtgtgtgtgtgtgtgtgtgtgtgtgtgtgtgtgt

SEQ ID NO: 13 - DNA, 32 bp, synthetic construct
  source 1..32: mol_type = other DNA
  modified_base 28: mod_base = OTHER; note = uracil
  modified_base 24: mod_base = OTHER; note = uracil
  modified_base 22: mod_base = OTHER; note = uracil
cgcgacggacggaaagacccctatccgtcgcg

SEQ ID NO: 14 - DNA, 47 bp, synthetic construct
  source 1..47: mol_type = other DNA
gactggtcattcaaaggggcttggccttgttcgttttacggaatatg

SEQ ID NO: 15 - DNA, 47 bp, synthetic construct
  source 1..47: mol_type = other DNA
catattccgtaaaacgaacaaggccaagcccctttgaatgaccagtc

SEQ ID NO: 16 - DNA, 45 bp, synthetic construct
  source 1..45: mol_type = other DNA
ccaagccattcagggcccttgcccttgttacatttgcagatgatc

SEQ ID NO: 17 - DNA, 45 bp, synthetic construct
  source 1..45: mol_type = other DNA
gatcatctgcaaatgtaacaagggcaagggccctgaatggcttgg

SEQ ID NO: 18 - DNA, 39 bp, synthetic construct
  source 1..39: mol_type = other DNA
gtgtgactgcaaacttcctaattctgagcaaagccaaga

SEQ ID NO: 19 - DNA, 39 bp, synthetic construct
  source 1..39: mol_type = other DNA
tcttggctttgctcagaattaggaagtttgcagtcacac

SEQ ID NO: 20 - DNA, 29 bp, synthetic construct
  source 1..29: mol_type = other DNA
gatgaactttggtgagttcagcattaatc

SEQ ID NO: 21 - DNA, 29 bp, synthetic construct
  source 1..29: mol_type = other DNA
gattaatgctgaactcaccaaagttcatc

SEQ ID NO: 22 - DNA, 48 bp, synthetic construct
  source 1..48: mol_type = other DNA
tttggtgcttcagcattaatccaggcatgatggctgccgcccaggcag

SEQ ID NO: 23 - DNA, 49 bp, synthetic construct
  source 1..49: mol_type = other DNA
ctgcctgggcggcagccatcatgcctggattaatgctgaacgcaccaaa

SEQ ID NO: 24 - DNA, 39 bp, synthetic construct
  source 1..39: mol_type = other DNA
gccgcccaggcagcactaaagagcagttggggtatgatg

SEQ ID NO: 25 - DNA, 39 bp, synthetic construct
  source 1..39: mol_type = other DNA
catcataccccaactgctctttagtgctgcctgggcggc

SEQ ID NO: 26 - DNA, 35 bp, synthetic construct
  source 1..35: mol_type = other DNA
ctctaattctggtgcaacaattggttggggatcag

SEQ ID NO: 27 - DNA, 35 bp, synthetic construct
  source 1..35: mol_type = other DNA
ctgatccccaaccaattgttgcaccagaattagag

SEQ ID NO: 28 - RNA, 35 nt, synthetic construct
  source 1..35: mol_type = other RNA
acacacacacacacacacacacacacacacacaca

SEQ ID NO: 29 - RNA, 11 nt, synthetic construct
  source 1..11: mol_type = other RNA
gtgtgatgaat

SEQ ID NO: 30 - RNA, 12 nt, synthetic construct
  source 1..12: mol_type = other RNA
gtgtgaatgaat

(19) United States
(12) Patent Application Publication
(10) Pub. No.: US 2023/0125788 A1
(43) Pub. Date: Apr. 27, 2023

(54) TDP-43 MODULATING AGENTS AND USES THEREOF

(71) Applicants: Timothy Miller, St. Louis, MO (US); Hunter Patterson, St. Louis, MO (US); Kathleen Schoch, St. Louis, MO (US); Yuna Ayala, St. Louis, MO (US); Zachary Grese, St. Louis, MO (US)

(72) Inventors: Timothy Miller, St. Louis, MO (US); Hunter Patterson, St. Louis, MO (US); Kathleen Schoch, St. Louis, MO (US); Yuna Ayala, St. Louis, MO (US); Zachary Grese, St. Louis, MO (US)

(73) Assignees: Washington University, St. Louis, MO (US); Saint Louis University, St. Louis, MO (US)

(21) Appl. No.: 18/049,903

(22) Filed: Oct. 26, 2022

Related U.S. Application Data
(60) Provisional application No. 63/271,999, filed on Oct. 26, 2021.

(51) Int. Cl. C12N 15/113; C12Q 1/6811; A61P 25/28

(52) U.S. Cl. CPC C12N 15/113; C12Q 1/6811; A61P 25/28

(57) ABSTRACT

Among the various aspects of the present disclosure is the provision of a method of treating a neurodegenerative disease in a subject in need thereof comprising administering a therapeutically effective amount of a TAR DNA-binding protein (TDP-43) modulating agent comprising an RNA oligonucleotide.

### CROSS-REFERENCE TO RELATED APPLICATIONS

This application claims priority from U.S. Provisional Application Ser. No. 63/271,999 filed on 26 Oct. 2021, which is incorporated herein by reference in its entirety.

### STATEMENT REGARDING FEDERALLY SPONSORED RESEARCH OR DEVELOPMENT

This invention was made with government support under W81XWH-20-1-0241 awarded by the Department of Defense. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention (file name 019930-US-NP_Replacement_Sequence_Listing.xml created on 12 Jan. 2023; 68,511 bytes). The subject matter of the Sequence Listing is incorporated herein by reference in its entirety.

### FIELD OF THE INVENTION

The present disclosure generally relates to RNA oligonucleotides targeting TAR DNA binding protein (TDP-43) and their use in treating neurodegenerative disorders.

### SUMMARY OF THE INVENTION

Among the various aspects of the present disclosure is the provision of methods and agents for treating neurodegenerative disorders linked to TDP-43 aggregation.

Other objects and features will be in part apparent and in part pointed out hereinafter.

In one aspect, a method of preventing or reducing aggregation, misfolding, or intracellular seeding of TAR DNA-binding protein (TDP-43) in a subject in need thereof is provided. The method comprises administering a therapeutically effective amount of a synthetic oligonucleotide having TDP-43 binding activity.

In some embodiments, the synthetic oligonucleotide comprises at least about ten nucleotides and is at least 50% identical to SEQ ID NO: 1, SEQ ID: NO: 2, SEQ ID NO: 3, SEQ ID NO: 4, or SEQ ID NO: 5 or is at least 50% identical to a corresponding, reverse, complement, or reverse-complement nucleotide sequence of SEQ ID NO: 1, SEQ ID NO: 2, SEQ ID NO: 3, SEQ ID NO: 4, or SEQ ID NO: 5.

In some embodiments, the synthetic oligonucleotide comprises SEQ ID NO: 1, SEQ ID: NO: 2, SEQ ID NO: 3, SEQ ID NO: 4, or SEQ ID NO: 5 or comprises a corresponding, reverse, complement, or reverse-complement nucleotide sequence of SEQ ID NO: 1, SEQ ID NO: 2, SEQ ID NO: 3, SEQ ID NO: 4, or SEQ ID NO: 5.

In some embodiments, the synthetic oligonucleotide comprises at least about 6, about 12, about 18, or about 30 (GU) repeats.

In some embodiments, the synthetic oligonucleotide comprises at least two TDP-43 binding sites.

In some embodiments, the subject has or is suspected of having a neurodegenerative disease, disorder, or condition. In further embodiments, the neurodegenerative disease, disorder, or condition is Amyotrophic Lateral Sclerosis (ALS), frontotemporal dementia (FTD or FTLD-TDP-43), limbic-predominant age-related TDP-43 encephalopathy (LATE), Alzheimer's disease, multisystem proteinopathy, or chronic traumatic encephalopathy.

In some embodiments, the synthetic oligonucleotide is a modified synthetic oligonucleotide comprising one or more backbone, sugar moiety, or nucleic base modifications.

In some embodiments, the modified synthetic oligonucleotide comprises a 2′-O-methyl base, 2′-O-methoxyethyl base, or phosphorothioate bond modification.

In some embodiments, the modified synthetic oligonucleotide comprises at least about ten nucleotides and is at least 50% identical to SEQ ID NO: 6, SEQ ID: NO: 7, SEQ ID NO: 8, or SEQ ID NO: 9, or is at least 50% identical to a corresponding, reverse, complement, or reverse-complement nucleotide sequence of SEQ ID NO: 6, SEQ ID NO: 7, SEQ ID NO: 8, or SEQ ID NO: 9. In further embodiments, the modified synthetic oligonucleotide comprises SEQ ID NO: 6, SEQ ID: NO: 7, SEQ ID NO: 8, or SEQ ID NO: 9, or comprises a corresponding, reverse, complement, or reverse-complement nucleotide sequence of SEQ ID NO: 6, SEQ ID NO: 7, SEQ ID NO: 8, or SEQ ID NO: 9.

In another aspect, a pharmaceutical composition is provided. The pharmaceutical composition comprises an isolated oligonucleotide having TDP-43 binding activity and at least 80% identity to a sequence selected from one of SEQ ID NO: 1, SEQ ID NO: 2, SEQ ID NO: 3, SEQ ID NO: 4, or SEQ ID NO: 5.

In yet another aspect, a method of treating a neurodegenerative disease, disorder, or condition in a subject in need thereof is provided. The method comprises administering a therapeutically effective amount of a synthetic oligonucleotide having TDP-43 binding activity.

In some embodiments, the synthetic oligonucleotide comprises at least about ten nucleotides and is at least 50% identical to SEQ ID NO: 1, SEQ ID: NO: 2, SEQ ID NO: 3, SEQ ID NO: 4, or SEQ ID NO: 5 or is at least 50% identical to a corresponding, reverse, complement, or reverse-complement nucleotide sequence of SEQ ID NO: 1, SEQ ID NO: 2, SEQ ID NO: 3, SEQ ID NO: 4, or SEQ ID NO: 5.

In some embodiments, the synthetic oligonucleotide comprises at least about 6, about 12, about 18, or about 30 (GU) repeats.

In some embodiments, the neurodegenerative disease, disorder, or condition is Amyotrophic Lateral Sclerosis (ALS), frontotemporal dementia (FTD or FTLD-TDP-43), limbic-predominant age-related TDP-43 encephalopathy (LATE), Alzheimer's disease, multisystem proteinopathy, or chronic traumatic encephalopathy.

In some embodiments, the synthetic oligonucleotide is a modified synthetic oligonucleotide comprising one or more backbone, sugar moiety, or nucleic base modifications. In further embodiments, the modified synthetic oligonucleotide comprises a 2′-O-methyl base, 2′-O-methoxyethyl base, or phosphorothioate bond modification. In further embodiments, the modified synthetic oligonucleotide comprises at least about ten nucleotides and is at least 50% identical to SEQ ID NO: 6, SEQ ID: NO: 7, SEQ ID NO: 8, or SEQ ID NO: 9, or is at least 50% identical to a corresponding, reverse, complement, or reverse-complement nucleotide sequence of SEQ ID NO: 6, SEQ ID NO: 7, SEQ ID NO: 8, or SEQ ID NO: 9. In further embodiments, the modified synthetic oligonucleotide comprises SEQ ID NO: 6, SEQ ID: NO: 7, SEQ ID NO: 8, or SEQ ID NO: 9, or comprises a corresponding, reverse, complement, or reverse-complement nucleotide sequence of SEQ ID NO: 6, SEQ ID NO: 7, SEQ ID NO: 8, or SEQ ID NO: 9.

### DESCRIPTION OF THE DRAWINGS

Those of skill in the art will understand that the drawings, described below, are for illustrative purposes only. The drawings are not intended to limit the scope of the present teachings in any way.

FIG. 1(A-B) depicts an exemplary embodiment showing RNA binding promotes liquid-like properties of TDP-43 in the dense phase in accordance with the present disclosure. FIG. 1A shows condensates formed by purified TDP-43 at 150 mM of salt observed by brightfield microscopy at different time points. FIG. 1B shows TDP-43 condensates without RNA control or in the presence of A(GU)₆ (8 μM), A(GU)₁₈ (2.6 μM), or A(CA)₆ (8 μM) (see e.g., TABLE 1) at 150 mM of NaCl (4 μM TDP-43). Representative images for three independent experiments with two different protein preparations. Scale bars, 10 μm.

FIG. 2 contains a graph and images showing RNA-driven LLPS of TDP-43 observed by fluorescence recovery after photobleaching (FRAP). FRAP of TDP-43-A(GU)₆ RNA condensates formed after mixing 4 μM TDP-43 with 8 μM A(GU)₆ at 150 mM of NaCl. Mean values and SD from three biological replicates comprising 14 bleached droplets, using two different protein purifications. Scale bar 2.5 μm.

FIG. 3 depicts an exemplary embodiment showing RNA binding promotes liquid-like properties of TDP-43 in the dense phase in accordance with the present disclosure. RNA binding-deficient TDP-43 mutants generated by site-directed substitutions in RRM1 (F2L) or in both RRM domains (F4L) and substitution of RRM1 and RRM2 with monomeric GFP (N-GFP-C). Phase separation of the mutants without added RNA (control) or in the presence A(GU)₆ (8 μM), A(GU)₁₈ (2.6 μM), or A(CA)₆ (8 μM) at 150 mM of NaCl (4 μM protein). Representative images for three independent experiments with two different protein preparations. Scale bars, 10 μm.

FIG. 4 contains graphs showing TDP-43 binding to (GU)₆ RNA analyzed by fluorescence anisotropy. Fluorescence anisotropy of different FITC-labeled A(GU)₆ concentrations titrated with increasing TDP-43 WT and mutants (0-65 nM). Each point is the average of >3 technical and 3 biological replicates, respectively (SEM). Solid lines represent the best global fit to calculate the apparent dissociation constant, K_{d,app} (see e.g., TABLE 2).

FIG. 5(A-B) depicts an exemplary embodiment showing RNA composed of multiple binding sites potently induces TDP-43 phase separation in accordance with the present disclosure. FIG. 5A is a table showing RNA oligonucleotide sequences composed of multiple TDP-43-binding sites, A(GU)₁₈, CLIP34 and (AUG12)₃, NS, or A(CA)₁₈ Sequence in boxes separate regions that accommodate one TDP-43 molecule, if known. NS, nonspecific control RNA. FIG. 5B contains images showing TDP-43 condensates in the presence of the different RNA sequences. TDP-43 and RNA oligonucleotide concentration is 4 μM and 3.9 μM, respectively. Representative images for three biological replicates using two different protein preparations. Scale bars, 10 μm.

FIG. 6 is an image showing RNA-driven TDP-43 phase separation is sequence dependent. Droplets observed by brightfield and fluorescence microscopy when Oregon green-labeled TDP-43 was mixed with no RNA control or Alexa 594-labeled A(GU)₁₈.

FIG. 7(A-B) is a series of images showing GU-rich RNA specifically modulates TDP-43 LLPS and TDP-43 does not depend on the N-terminal SUMO tag. FIG. 7A shows untagged TDP-43 was recovered after cleaving the N-terminal SUMO tag by Ulpl, and mixed with either control, 3.9 μM A(GU)₁₈, or 3.9 μM A(CA)₁₈ at 250 mM NaCl. The concentration of TDP-43 recovered after cleavage was estimated between 0.25-0.50 μM. Representative images of 3 biological replicates using 2 different protein preparations. Scale bar 10 μm. FIG. 7B shows TDP-43 LLPS observed by brightfield microscopy in the presence of A(GU)₁₂ and two sequences of nonmodified RNA, A(GU)₁₂ and (CA)₁₂ at 250 mM NaCl, 4 μM TDP-43. Lower panels show TDP-43 LLPS in the presence of single stranded DNA that binds TDP-43 with >100-fold lower affinity than GU-repeat RNA, (GT)₁₈, non-specific single stranded DNA NS (see e.g., TABLE 1), and heparin. Representative images for 3 biological replicates using 2 different protein preparations. RNA at 250 mM of salt. Representative images for three biological replicates using two protein preparations. Scale bars, 10 μm.

FIG. 8 contains an image and schematic showing CLIP34 RNA binds multiple TDP-43 molecules. Electromobility shift assays (EMSA) or band shift analysis of CLIP34 RNA binding to TDP-43 seen upon separation of complexes by polyacrylamide gel electrophoresis. Single stranded CLIP34 oligonucleotide (see e.g, FIG. 5A) labeled with a 700 nm IR moiety at the 5′ end (10 nM) was incubated with full-length purified TDP-43 at increasing concentrations (0 to 7 μM) for 5 min on ice and 25 min at room temperature in binding buffer (150 mM NaCl; 10 mM Tris pH 8; 2 mM MgCl2; 5% glycerol; 1 mM DTT). Electrophoresis of 6% acrylamide gels was performed at 100 V and images were acquired using the LI-COR Odyssey platform.

FIG. 9(A-C) depicts an exemplary embodiment showing RNA composed of multiple binding sites potently induces TDP-43 phase separation in accordance with the present disclosure. FIG. 9A is a graph showing LLPS measured by turbidity in the same experimental conditions as FIG. 5B Mean and SD from three biological replicates with two different protein preparations. FIG. 9B contains images showing lysate from HEK 293 cells expressing a mEGFP-tagged copy of either wildtype (WT) or F147/149/229/231L (F4L) TDP-43 was added to a mixture of recombinant WT or F4L TDP-43 (5.3 μM, 10% Cy3-labeled protein) and A(GU)₆ (22.8 μM), A(CA)₁₈ (7.6 μM), A(GU)₁₈ (7.6 μM), or no RNA control at 250 mM of NaCl. Droplets were imaged by brightfield and fluorescence microscopy. Representative images for three biological replicates using two protein preparations. Scale bars, 10 μm. FIG. 9C is a graph showing LLPS measured by turbidity in the same experimental conditions as FIG. 9B. Mean and SD of four biological replicates using two recombinant protein preparations. Analyzed by one-way ANOVA (F(7,24)=69.30, P<0.0001). Sidak's multiple comparisons test was used to compare selected groups. *P≤0.05, ****P≤0.0001.

FIG. 10(A-C) depicts an exemplary embodiment showing RNA with multiple binding sites modulates TDP-43 phase properties in accordance with the present disclosure. FIG. 10A contains images showing liquid droplets observed by brightfield and fluorescence microscopy when Oregon green labeled TDP-43 (4 μM, 10% labeled protein) was mixed with no RNA control or increasing A(GU)₁₈ RNA concentration at 250 mM of salt. Representative images from three biological replicates using two different protein preparations. Scale bars, 10 μm. FIG. 10B is a graph showing area of droplets plotted at different A(GU)₁₈ concentrations. Mean and SD of >300 droplets from three biological replicates using two different protein preparations. FIG. 10C is a graph showing TDP-43 concentration in the light phase measured as a function of A(GU)₁₈ or A(CA)₁₈ concentration measured by Bradford protein assay. Mean and SD from ≥6 biological replicates using two different protein preparations (250 mM of NaCl, 4 μM TDP-43).

FIG. 11(A-B) depicts an exemplary embodiment showing RNA-driven TDP-43 phase separation is sequence dependent in accordance with the present disclosure. FIG. 11A contains images showing liquid droplets observed by brightfield and fluorescence microscopy when Oregon green-labeled TDP-43 (4 μM, 10% labeled protein) was mixed with no RNA control or increasing A(GU)₁₈ at 250 mM of salt. FIG. 11B contains images showing liquid droplets observed by brightfield and fluorescence microscopy when Oregon green-labeled TDP-43 (4 μM, 10% labeled protein) was mixed with increasing A(CA)₁₈ RNA concentration at 250 mM of salt. Representative images from three biological replicates using two different protein preparations. Scale bars, 10 μm.

FIG. 12(A-B) depicts an exemplary embodiment showing RNA with multiple binding sites modulates TDP-43 phase properties in accordance with the present disclosure. FIG. 12A contains images showing phase separation of TDP-43 (4 μM) at increasing salt concentration without RNA or in the presence of increasing A(GU)₁₈ concentration. Scale bars, 10 μm. Inlay added to top right panel for increased visibility, scale bar 1 μm. FIG. 12B is a phase diagram derived from FIG. 12A, denoting either no droplets or droplet formation for each condition.

FIG. 13(A-B) depicts an exemplary embodiment showing increasingly high concentrations of GU repeat RNA drive TDP-43 out of liquid-liquid phase separation in accordance with the present disclosure. FIG. 13A contains images showing droplets observed by brightfield when TDP-43 was mixed with no RNA control or increasing concentrations of A(GU)₁₅ RNA at 250 mM of NaCl. Representative images for three biological replicates using two protein preparations. Scale bars, 10 μm. FIG. 13B is a graph showing LLPS measured by turbidity in the same experimental conditions as FIG. 13A.

FIG. 14 depicts an exemplary embodiment showing TDP-43 phase separation is regulated by the number of binding sites on RNA in accordance with the present disclosure. TDP-43 droplets observed by brightfield and fluorescence microscopy are shown when Oregon green labeled TDP-43 (4 μM, 10% labeled protein) was mixed with no RNA control or GU-repeat RNA oligonucleotides of increasing length at 250 mM of salt. As indicated, RNA concentration varied to maintain a constant total number of TDP-43-binding sites. Scale bars, 10 μm. Mean and SD from three biological replicates using two different protein preparations.

FIG. 15(A-B) is a series of images showing RNA induction of TDP-43 phase separation is length dependent and sequence specific. FIG. 15A shows RNA composed of GU repeats and FIG. 15B shows RNA composed of CA repeats mixed with TDP-43 (4 μM) at 250 mM of NaCl. To maintain the concentration of binding modules, the concentration of the separate oligonucleotides varied as follows: 4 μM A(GU)₆ and A(CA)₆, 1.3 μM A(GU)₁₈ and A(CA)₁₈, and 1 μM A(GU)₂₄ and A(CA)₂₄. Imaged by brightfield and fluorescence microscopy. Representative images of three biological replicates using two different protein preparations. Scale bars, 10 μm.

FIG. 16 is a graph showing GU-rich RNA promotes TDP-43 LLPS in a length dependent manner. Spontaneous TDP-43 phase separation in the presence of no RNA control and RNA oligonucleotides with increasing number of GU repeats at 250 mM NaCl. LLPS was measured by turbidity and plotted as a function of GU dinucleotide repeat number. RNA oligonucleotide concentration varied to maintain constant concentration of putative binding sites (4 μM A(GU)₆, 2.6 μM A(GU)₉, 2 μM A(GU)₁₂, 1.6 μM A(GU)₁₆, 1.3 μM A(GU)₁₈, 1 μM A(GU)₂₄, 0.8 μM A(GU)₃₀). Mean values and SD from 3 biological replicates using 2 different protein preparations.

FIG. 17(A-B) depicts an exemplary embodiment showing TDP-43 phase separation is regulated by the number of binding sites on RNA in accordance with the present disclosure. FIG. 17A is a graph showing droplet area quantified as a function of GU-repeat length. Mean and SD of >150 droplets from three biological replicates using three different protein preparations. FIG. 17B is a graph showing TDP-43 concentration in the light phase (Cₒᵤₜ) as a function of GU- or CA-repeat length quantified by Bradford protein assay. Mean and SD from ≥5 biological replicates using two different protein preparations. (250 mM of NaCl, 4 μM TDP-43).

FIG. 18(A-B) is an exemplary embodiment depicting RNA-induced phase separation requires multivalent interactions mediated by TDP-43 domains in accordance with the present disclosure. FIG. 18A is a schematic showing full-length TDP-43, mutant, and deletion fragments targeting regions that mediate self-assembly. Amino acid residue positions are indicated for each. FIG. 18B contains images showing phase separation of full-length and TDP-43 variants (4 μM) mixed with A(GU)₃₀ RNA (0.8 μM) or no RNA control at 250 mM of NaCl observed by brightfield microscopy. Representative images for three biological replicates using two different protein preparations. Scale bars, 10 μm.

FIG. 19 contains graphs showing TDP-43 binding to (GU)₆ RNA analyzed by fluorescence anisotropy. Fluorescence anisotropy of different FITC-labeled A(GU)₆ concentrations titrated with increasing mutant TDP-43 (0-65 nM). Each point is the average of >3 technical and 3 biological replicates, respectively (SEM). Solid lines represent the best global fit to calculate the apparent dissociation constant, K_{d,app} (see e.g., TABLE 2).

FIG. 20 depicts an exemplary embodiment showing ALS-linked TDP-43 mutations A321G and M337V impair RNA-driven phase separation in accordance with the present disclosure. TDP-43 ALS-linked amino acid substitutions K181E, A315T/E, A321G, Q331K, M337V, and A382T are shown. Boxed in the C-terminal domain is the α-helical structure within the conserved region (a.a. 320-343). Droplets formed by TDP-43 wild-type (VVT) and the different mutants (4 μM) observed by brightfield microscopy in the presence of no RNA control or A(GU)₁₈ RNA (3.9 μM) at 250 mM of NaCl. Mutations A321G and M337V, showing decreased liquidity of the condensates in the presence of A(GU)₁₈, are boxed in red. Representative images for three biological replicates using three different protein preparations of WT and M337V, 2 preparations of A321G, Q331K, K181E and one preparation of A315T/E and A382T. Scale bars, 10 μm.

FIG. 21 contains graphs showing ALS-linked TDP-43 mutants binding to (GU)₆ RNA analyzed by fluorescence anisotropy. Fluorescence anisotropy of different FITC-labeled A(GU)₆ concentrations titrated with increasing mutant TDP-43 (0-65 nM). Each point is the average of >3 technical and 3 biological replicates, respectively (SEM). Solid lines represent the best lobal fit to calculate the apparent dissociation constant, K_{d,app} (see e.g., TABLE 2).

FIG. 22 depicts an exemplary embodiment showing ALS-linked TDP-43 mutations A321G and M337V impair RNA-driven phase separation in accordance with the present disclosure. Phase separation of WT (4 μM, 10% Oregon green-labeled protein) and M337V (4 μM, 10% Cy-3-labeled protein) observed by brightfield and fluorescence microscopy in the presence of A(GU)₁₈ (3.9 μM) at 250 mM of NaCl is shown. The middle panel shows mixing of these WT (2 μM) and M337V (2 μM) samples. Representative images for three biological replicates using two protein preparations. Scale bars, 10 μm.

FIG. 23(A-B) depicts an exemplary embodiment showing RNA binding is a key driver of TDP-43 phase separation maintaining liquid properties of the dense state. FIG. 23A shows binding of TDP-43 to GU-rich RNA promotes liquid properties of TDP-43 condensates, as shown by the fusion of small condensates into larger droplets. This fluidity is impaired by the loss of RNA-binding affinity or disease-linked mutations that alter phase separation, such as M337V and A321G, which could explain increased TDP-43 aggregation. FIG. 23B shows RNA molecules that bind multiple TDP-43 molecules provide a scaffold and increase multivalent interactions, including N- and C-terminal domains, creating a meshwork that results in phase separation. In contrast, nonspecific RNA fails to sufficiently concentrate TDP-43 to generate the multivalent network necessary for condensation. Scale bars, 10 μm.

FIG. 24(A-C) is an exemplary embodiment depicting GU-rich RNA inhibits the aggregation of purified TDP-43 in accordance with the present disclosure. FIG. 24A is an image showing Immunoblotting upon extraction of soluble and insoluble fractions of purified TDP-43 at time 0 and after aggregation (Day 5) in the presence of A(GU)₆ RNA and control. FIG. 24B is a graph showing quantification of normalized insoluble TDP-43 from FIG. 24A after treatment with A(GU)₆ and control A(CA)₆ RNA. Means and SD of at least 3 independent replicates. Analyzed by one-way ANOVA. Dunett's multiple comparisons test was used to compare treatment groups to control. **P≤0.01. FIG. 24C is a table showing apparent dissociation constants measured in FIG. 4 for TDP-43 binding to GU-rich RNA and the control sequence A(CA)₆.

FIG. 25(A-C) depicts an exemplary embodiment showing GU-rich RNA oligonucleotides inhibit the aggregation of TDP-43 in human cells in accordance with the present disclosure. FIG. 25A is an immunoblot detecting endogenous TDP-43 in total HEK293 cell lysate or following fractionation into soluble and insoluble extracts. Cells were treated with control or proteotoxic stress conditions following transfection of A(GU)₆ or no RNA control. FIG. 25B is a graph showing quantification of TDP-43 aggregation under proteotoxic conditions, measured as insoluble/soluble ratio from FIG. 25A at increasing A(GU)₆ concentration or in the presence of non-specific control RNA (NS). Analysis includes at least 5 biological independent replicates by one-way ANOVA. Dunett's multiple comparisons test was used to compare treatment groups to control. **P≤0.01, ****P≤0.0001. FIG. 25C is a graph showing quantification of insoluble to soluble TDP-43 ratio under proteotoxic conditions in the presence of different GU-rich RNA sequences. A(CA)₆ RNA used as control. The values were normalized to the ratio of stress-treated cells in the absence of RNA. Analysis includes at least 5 biological independent replicates by one-way ANOVA. Dunett's multiple comparisons test was used to compare treatment groups to control. *P≤0.05, **P≤0.01, ***P≤0.001.

FIG. 26 contains a schematic and graph showing specific RNA binding is necessary to prevent TDP-43 aggregation by RNA. Four site specific mutations in the RNA binding domains (F4L) disrupt TDP-43 RNA binding activity. Quantification of TDP-43 aggregation from HEK293 cells stably expressing hemagglutinin (HA) tagged TDP-43, WT or F4L. The insoluble/soluble ratio of HA-TDP-43 transgene and endogenous TDP-43 from cells treated with proteotoxic stress, following transfection with A(GU)₆ RNA. Values were normalized to no RNA control. Mean of 8 biological independent replicates and analyzed by one-way ANOVA. Sidak's multiple comparisons test was used to compare treatment groups. *P≤0.05, **P≤0.01.

FIG. 27 contains a schematic and graph showing RNA inhibits TDP-43 intracellular seeding. HEK293 cells stably expressing mCherry tagged TDP-43, carrying mutations in the nuclear localization signal. Cells are treated with pre-formed aggregates of purified TDP-43. The seeds are internalized and colocalize with de novo cellular TDP-43 inclusions. Cells were treated with control, purified soluble TDP-43, TDP-43 aggregates formed in the presence and absence of A(GU)₆. Quantification of pTDP-43-positive cells relative to control. Mean of four independent replicates and S.D. analyzed by one-way ANOVA, *P=0.01, ***P=3×10⁻⁴.

FIG. 28 depicts an exemplary embodiment showing TDP-43 binding affinity to synthetic RNA is dramatically modulated by chemical RNA modifications in accordance with the present disclosure. Fluorescence anisotropy was performed for modified CLIP-34 (e.g., antisense oligonucleotide), unmodified CLIP-34, modified A(GU)₆, and unmodified A(GU)₆ incubated with wild-type TDP-43.

FIG. 29(A-B) depicts an exemplary embodiment showing identification of additional RNA oligonucleotides that promote TDP-43 solubility in accordance with the present disclosure. FIG. 29A is a schematic showing the RNA sequence CLIP34. FIG. 29B contains images showing CLIP34 specifically increases the liquid properties of TDP-43 condensates and prevents formation of solid-like fibrils.

FIG. 30 depicts an exemplary embodiment showing transfection of RNA oligonucleotides decrease TDP-43-mediated neurotoxicity. TDP43-expressing primary rodent neurons were transfected with control or CLIP34 ASOs. CLIP34 ASOs displayed distinct effects on neuron survival.

FIG. 31(A-D) depicts an exemplary embodiment showing RNA—driven condensation increases TDP-43 nuclear retention in accordance with the present disclosure. FIG. 31A contains images showing WT TDP-43 is predominantly nuclear but exits to the cytoplasm when it is not found in large assemblies. Increased TDP-43 levels in the cytoplasm is linked to pathology. FIG. 31B is a schematic showing the location of the tested mutations in the TDP-43 transcript. FIG. 31C is a graph showing RNA-driven formation of large TDP-43 complexes in the nucleus helps retain TDP-43 in the nucleus. FIG. 31D is a graph showing Overexpression of a specific RNA transcript increases TDP-43 nuclear retention.

### DETAILED DESCRIPTION OF THE INVENTION

The present disclosure is based, at least in part, on the discovery that specific RNA oligonucleotides may be used to prevent TDP-43 aggregation and decrease neurotoxicity.

TDP-43 is an RNA binding protein that is involved in multiple neurodegenerative diseases including amyotrophic lateral sclerosis and Alzheimer's disease. Aggregation and misfolding of TDP-43 is a pathological hallmark of these diseases. As described in Examples 1 and 2, RNA binding to TDP-43 inhibits aggregation. Furthermore, specific sequences, for example repetitive GU sequences (e.g., such as repeats between 1 and 50 GU repeats), may be particular effective for inhibition of aggregation. As described herein, modified RNA, e.g., antisense oligonucleotides or ASOs, with the same sequence may be used to test whether the ASO can inhibit TDP-43 aggregation and behavioral dysfunction in a mouse model of mutant TDP-43. These experiments are currently underway.

Currently, there are no effective ways to inhibit TDP-43 in vivo. Defining optimal sequences in vitro and translating to in vivo is the beginning of defining an effective TDP-43 focused therapy.

Synthetic Oligonucleotides Having TDP-43 Binding Activity

The present disclosure provides for synthetic oligonucleotides having TDP-43 binding activity. Aberrant self-association of TDP-43 may lead to protein aggregation and pathology associated with numerous neurodegenerative disorders. As described herein, RNA binding plays a central role in modulating TDP-43 condensation while maintaining protein solubility TDP-43 aggregation and misfoldin. Surprisingly, synthetic oligonucleotides having TDP-43 binding activity were shown to reduce TDP-43 aggregation or misfolding or decrease neurotoxicity (see e.g., Examples 1-3). A synthetic oligonucleotide as described herein refers to an oligonucleotide that is not naturally occurring.

The synthetic oligonucleotide of the present disclosure may be any oligonucleotide that binds to a TDP-43 protein and effectively reduces or prevents TDP-43 protein aggregation, misfolding, or intracellular seeding. For example, the synthetic oligonucleotide may bind to at least one RNA recognition motif (RRM) of TDP-43, such as RRM1, RRM2, or both. As another example, the synthetic oligonucleotide may bind to or interact with an N-terminal domain (NTD) or carboxy-terminal domain (CTD) of TDP-43. A single synthetic oligonucleotide of the present disclosure may bind to more than one TDP-43 protein, e.g., comprise multiple TDP-43 binding sites.

In some embodiments, the synthetic oligonucleotide having TDP-43 binding activity comprises

(i) AGU₁₈:
(SEQ ID NO: 1)
AGUGUGUGUGUGUGUGUGUGUGUGUGUGUGUGUGUGU;
(ii) CLIP34:
(SEQ ID NO: 2)
GAGAGAGCGCGUGCAGAGACUUGGUGGUGCAUAA;
(iii) (AUG12)₃:
(SEQ ID NO: 3)
GUGUGAAUGAAUGUGUGAAUGAAUGUGUGAAUGAAU;
(iv) AGU₆:
(SEQ ID NO: 4)
AGUGUGUGUGUGU;
or
(v) AGT₆:
(SEQ ID NO: 5)
AGTGTGTGTGTGT.

In some embodiments, the synthetic oligonucleotide comprises at least about ten nucleotides and is at least about 50% identical to SEQ ID NO: 1, SEQ ID NO: 2, SEQ ID NO: 3, SEQ ID NO: 4, or SEQ ID NO: 5 or is at least about 50% identical to a corresponding, reverse, complement, or reverse-complement nucleotide sequence of SEQ ID NO: 1, SEQ ID NO: 2, SEQ ID NO: 3, or SEQ ID NO: 4., or SEQ ID NO: 5.

The present disclosure also provides for a modified synthetic oligonucleotide, e.g., a synthetic oligonucleotide comprising at least one modification to the backbone, a sugar moiety, or a nucleic base comprised in the synthetic oligonucleotide. Such modifications may be included to, for example, improve pharmacological properties of the synthetic oligonucleotide such as binding affinity for TDP-43 (see e.g., Example 3) or improve resistance to endonucleases. For example, modifications to the backbone may include a phosphorothioate (PS) bond modification, phosphoramidate bond modification, or thiophosphoramidate bond modification. As another example, modifications to the sugar moiety may include 2′-O-methyl, 2′-O-methoxyethyl (MOE), 2′-fluoro locked nucleic acid (LNA), ethylene-bridged nucleic acid (ENA), or (S)-constrained ethyl (cEt). As yet another example, modifications to the nucleic base may include 5-Methylcytidine, 5-Methyluridine, or abasic RNA.

As described herein, it was surprisingly found TDP-43 binding affinity to synthetic RNA was dramatically modulated by chemical RNA modifications (see e.g., Example 3).

In some embodiments, the the modified synthetic oligonucleotide comprises:

(i) AGU₆ 2OMe modified:
(SEQ ID NO: 6)
mA*mG*mU*mG*mU*mG*mU*mG*mU*mG*mU*mG*mU
(ii) CLIP-34 2OMe modified:
(SEQ ID NO: 7)
mG*mA*mG*mA*mG*mA*mG*mC*mG*mC*mG*mU*mG*mC*mA*
mG*mA*mG*mA*mC*mU*mU*mG*mG*mU*mG*mG*mU*mG*mC*
mA*mU*mA*mA
(iii) AGT₆ MOE modified:
(SEQ ID NO: 8)
/52MOErA/*/i2MOErG/i2MOErT/*/i2MOErG/i2MOErT/*/
i2MOErG/*/i2MOErT/*/i2MOErG/*/i2MOErT/i2MOErG/*/
i2MOErT/i2MOErG/*/32MOErT/
(iv) AGU₆ mixed MOE OMe modified:
(SEQ ID NO: 9)
/52MOErA/*/i2MOErG/mU*/i2MOErG/mU*/i2MOErG/*mU*/
i2MOErG/*mU/i2MOErG/*mU/i2MOErG/*mU

The * represents a phosphorothioate bond modification to avoid degradation by exonucleases.
The /52MOEr/ represents 5′ 2-MethoxyEthoxy (2′-O-methoxy-ethyl).
The /i2MOEr/ represents internal 2-MethoxyEthoxy (2′-O-methoxy-ethyl).
The /32MOEr/ represents 3′ 2-MethoxyEthoxy (2′-O-methoxy-ethyl).
The m represents 2′ O-methyl.

In some embodiments, the modified synthetic oligonucleotide comprises at least about ten nucleotides and is at least about 50% identical to SEQ ID NO: 6, SEQ ID NO: 7, SEQ ID NO: 8, or SEQ ID NO: 9, or is at least about 50% identical to a corresponding, reverse, complement, or reverse-complement nucleotide sequence of SEQ ID NO: 6, SEQ ID NO: 7, SEQ ID NO: 8, or SEQ ID NO: 9.

Diseases, Disorders, or Conditions Associated with TDP-43 Aggregation

The present disclosure provides for synthetic oligonucleotides that specifically bind to an RNA binding domain of TDP-43 to reduce aggregation of TDP-43. Synthetic oligonucleotides targeting TDP-43 can be used to treat or prevent diseases, disorders, or conditions associated with TDP-43 aggregation.

Diseases, disorders, or conditions associated with TDP-43 aggregation can be a neurodegenerative disease, disorder, or condition. As an example, a neurodegenerative disease, disorder, or condition can be Alzheimer's disease, amyotrophic lateral sclerosis (ALS), Alexander disease, Alpers' disease, Alpers-Huttenlocher syndrome, alpha-methylacyl-CoA racemase deficiency, Andermann syndrome, Arts syndrome, ataxia neuropathy spectrum, ataxia (e.g., with oculomotor apraxia, autosomal dominant cerebellar ataxia, deafness, and narcolepsy), autosomal recessive spastic ataxia of Charlevoix-Saguenay, Batten disease, beta-propeller protein-associated neurodegeneration, Cerebro-Oculo-Facio-Skeletal Syndrome (COFS), Corticobasal Degeneration, CLN1 disease, CLN10 disease, CLN2 disease, CLN3 disease, CLN4 disease, CLN6 disease, CLN7 disease, CLN8 disease, cognitive dysfunction, congenital insensitivity to pain with anhidrosis, dementia, familial encephalopathy with neuroserpin inclusion bodies, familial British dementia, familial Danish dementia, fatty acid hydroxylase-associated neurodegeneration, frontotemporal dementia (FTD), Gerstmann-Straussler-Scheinker Disease, GM2-gangliosidosis (e.g., AB variant), HMSN type 7 (e.g., with retinitis pigmentosa), Huntington's disease, infantile neuroaxonal dystrophy, infantile-onset ascending hereditary spastic paralysis, Huntington's disease (HD), infantile-onset spinocerebellar ataxia, juvenile primary lateral sclerosis, Kennedy's disease, Kuru, Leigh's Disease, Marinesco-Sjögren syndrome, Mild Cognitive Impairment (MCI), mitochondrial membrane protein-associated neurodegeneration, Motor neuron disease, Monomelic Amyotrophy, Motor neuron diseases (MND), Multiple System Atrophy, Multiple System Atrophy with Orthostatic Hypotension (Shy-Drager Syndrome), multiple sclerosis, multiple system atrophy, neurodegeneration in Down's syndrome (NDS), neurodegeneration of aging, Neurodegeneration with brain iron accumulation, neuromyelitis optica, neuronal ceroid lipofuscinosis (NCL), pantothenate kinase-associated neurodegeneration, Opsoclonus Myoclonus, prion disease, Progressive Multifocal Leukoencephalopathy, Parkinson's disease (PD), PD-related disorders, polycystic lipomembranous osteodysplasia with sclerosing leukoencephalopathy, prion disease, progressive external ophthalmoplegia, riboflavin transporter deficiency neuronopathy, Sandhoff disease, Spinal muscular atrophy (SMA), Spinocerebellar ataxia (SCA), Striatonigral degeneration, Transmissible Spongiform Encephalopathies (Prion Diseases), or Wallerian-like degeneration.

As another example, the neurodegenerative disease, disorder, or condition can be Amyotrophic Lateral Sclerosis (ALS), frontotemporal dementia (FTD), limbic-predominant age-related TDP-43 encephalopathy (LATE), Alzheimer's disease, multisystem proteinopathy, or chronic traumatic encephalopathy.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine), Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g.,

Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated in any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intranasal, inhalation (e.g., in an aerosol), implanted, intramuscular, intraperitoneal, intravenous, subcutaneous, intranasal, epidural, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating or preventing a neurodegenerative disease or disorder associated with TDP-43 aggregation in a subject in need thereof comprising administration of a therapeutically effective amount of a synthetic oligonucleotide having TDP-43 binding activity so as to reduce aggregation or restore function of TDP-43.

Methods described herein are generally performed on a subject in need thereof. A subject in need of the therapeutic methods described herein can be a subject having, diagnosed with, suspected of having, or at risk for developing a neurodegenerative disease or disorder associated with TDP-43 aggregation. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of a synthetic oligonucleotide having TDP-43 binding activity is, for example, that amount that would cause the desired therapeutic effect in a subject while minimizing undesired side effects. In various embodiments, an effective amount of a synthetic oligonucleotide having TDP-43 binding activity described herein can substantially reduce TDP-43 aggregation to inhibit neurodegeneration, reduce neurotoxicity, slow the progress of neurodegeneration, or limit the development of neurodegeneration.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a synthetic oligonucleotide having TDP-43 binding activity can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to increase progranulin protein expression.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a synthetic oligonucleotide having TDP-43 binding activity can occur as a single event or over a time course of treatment. For example, a synthetic oligonucleotide having TDP-43 binding activity can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for neurodegenerative diseases or disorders.

A synthetic oligonucleotide having TDP-43 binding activity can be administered simultaneously or sequentially with another agent, such as an antibiotic, an anti-inflammatory, or another agent. For example, a synthetic oligonucleotide having TDP-43 binding activity can be administered simultaneously with another agent, such as an antibiotic or an anti-inflammatory. Simultaneous administration can occur through administration of separate compositions, each containing one or more of a synthetic oligonucleotide having TDP-43 binding activity, an antibiotic, an anti-inflammatory, or another agent. Simultaneous administration can occur through administration of one composition containing two or more of a synthetic oligonucleotide having TDP-43 binding activity, an antibiotic, an anti-inflammatory, or another agent. A synthetic oligonucleotide having TDP-43 binding activity can be administered sequentially with an antibiotic, an anti-inflammatory, or another agent. For example, a synthetic oligonucleotide having TDP-43 binding activity can be administered before or after administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to an RNA oligonucleotide targeting TDP-43. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, or sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Specific RNA Interactions Promote TDP-43 Multivalent Phase Separation and Maintain Liquid Properties

The following example describes RNA oligonucleotides that bind to TDP-43 to modulate TDP-43 condensation and maintain protein solubility through sequence-specific interactions.

TDP-43 is an RNA-binding protein that forms ribonucleoprotein condensates via liquid-liquid phase separation (LLPS) and regulates gene expression through specific RNA interactions. Loss of TDP-43 protein homeostasis and dysfunction are tied to neurodegenerative disorders, mainly amyotrophic lateral sclerosis (ALS) and frontotemporal dementia. Alterations of TDP-43 LLPS properties may be linked to protein aggregation. However, the mechanisms regulating TDP-43 LLPS are ill-defined, particularly how TDP-43 association with specific RNA targets regulates TDP-43 condensation remains unclear. Shown herein is that RNA binding strongly promotes TDP-43 LLPS through sequence-specific interactions. RNA-driven condensation increases with the number of adjacent TDP-43-binding sites and is also mediated by multivalent interactions involving the amino and carboxy-terminal TDP-43 domains. The physiological relevance of RNA-driven TDP-43 condensation is supported by similar observations in mammalian cellular lysate. Importantly, TDP-43-RNA association was shown to maintain liquid-like properties of the condensates, which are disrupted in the presence of ALS-linked TDP-43 mutations. Altogether, RNA binding plays a central role in modulating TDP-43 condensation while maintaining protein solubility, and defects in this RNA-mediated activity may underpin TDP-43-associated pathogenesis.

### INTRODUCTION

The intracellular organization of RNA-binding proteins into ribonucleoprotein (RNP) granules is mediated by a process of condensation or phase separation. Based on evidence emerging over the last decade, these protein and RNA-rich granules are formed via liquid-liquid phase separation (LLPS) that often show dynamic, liquid-like properties. The high concentration of the components while retaining dynamic properties within the droplets may be essential to determine function, cellular organization, and rapid response to cellular stimuli. Formation of RNP granules is mediated by multivalent interactions, including RNA binding and self-assembly through different protein domains, such as low complexity regions. Although RNA is a fundamental component of RNP granules, how it regulates the biogenesis and metabolism of RNP condensates is not well understood. Recent studies suggest that RNA targets may modulate the LLPS properties of RNA-binding proteins through specific interactions. Numerous reports also suggest that defects in LLPS homeostasis are associated with disease, such as in the case of TDP-43 (TAR DNA-binding protein), FUS (Fused in sarcoma), and other RNA-binding proteins linked to neurodegeneration. Specifically, conversion of the condensates into fibrils or complexes with solid-like properties may lead to the accumulation of protein aggregates associated with pathology. Aggregation and loss of TDP-43 function are hallmarks of amyotrophic lateral sclerosis (ALS), frontotemporal dementia (FTD), and limbic-predominant age-related TDP-43 encephalopathy (LATE). In addition, TDP-43 inclusions are associated with multisystem proteinopathy (MSP) and other neurodegenerative disorders, including Alzheimer's disease (AD) and chronic traumatic encephalopathy (CTE).

TDP-43 is a multi-domain heterogeneous ribonucleoprotein (hnRNP) whose function is essential for the regulation of hundreds of mRNA transcripts to which it binds. The best-characterized TDP-43 function is regulation of splicing, however, TDP-43 also participates in different mechanisms of RNA processing and transport. For example, TDP-43 plays a more global role in gene regulation as an inhibitor of cryptic exon inclusion and regulates the alternative polyadenylation of >1,000 genes through direct. TDP-43 shows a strong preference for binding dinucleotide guanosine and uridine (GU) RNA motifs as seen in vitro and through the identification of coding and noncoding transcripts that bind TDP-43 in mouse brain and in human cells. Purified TDP-43 binds GU repeat sequences tightly through specific interactions that are evolutionarily conserved throughout higher eukaryotes. TDP-43 forms RNP granules in different cell types, including Cajal bodies and paraspeckles in the nucleus and is recruited to mRNA transport granules in neurons. TDP-43-positive granules may form under conditions of proteotoxic stress as nuclear or cytoplasmic stress granules. In addition, specific phosphorylation in RRM1 results in nucleolar localization, suggesting that posttranslational modifications may regulate TDP-43 RNP body dynamics.

To investigate the mechanisms underlying RNA-mediated assembly of TDP-43 RNP granules, which remain largely unknown, whether TDP-43 LLPS properties are modulated by RNA molecules depending on sequence and number of proximal protein-binding sites was investigated herein. Specific interactions of TDP-43 with RNA consisting of multiple binding sites readily promote phase separation into droplets that exhibit the liquid-like properties associated with LLPS. Moreover, specific RNA-binding increases the liquid properties of TDP-43 droplets. With this experimental paradigm in place, the impact of disease-associated TDP-43 mutations on RNA-mediated LLPS properties of TDP-43 was determined. It was found that specific mutations reduced the liquid properties of RNA-driven TDP-43 condensates. Importantly, specific RNA binding also triggered TDP-43 phase separation in mammalian cell lysate in the presence of multiple binding sites. Based on these observations, physiological RNA recruitment of TDP-43 may nucleate phase separation and, at the same time, prevent the conversion of the droplets into solid-like complexes.

Results

RNA Binding Increases the Liquid-Like Properties of TDP-43 Condensates

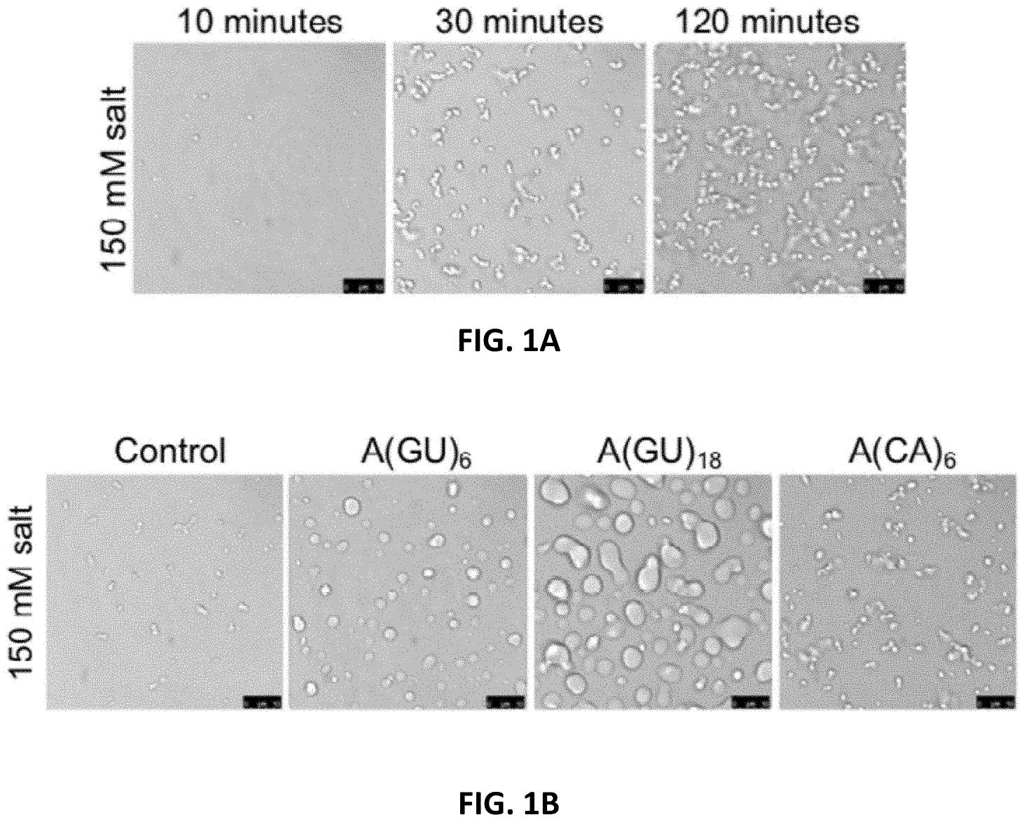

A major question in the field is whether TDP-43 condensation necessarily promotes protein aggregation, and what factors prevent TDP-43 loss of proteostasis in the condensed phase. It was previously shown that specific RNA binding inhibits TDP-43 aggregation. The chaperone-like activity of GU-rich RNA in maintaining TDP-43 solubility depends on specific interactions with the RNA-binding domains. Herein, it was investigated whether RNA binding increases TDP-43 solubility during phase separation. For these studies, reconstitution assays were established using purified TDP-43. Until recently, these experiments using full-length TDP-43 have been challenging, compared with studying related proteins, such as FUS and hnRNP A1, because the condensates rapidly convert into aggregates. Purification and assay conditions were established whereby TDP-43 formed small droplets at 150 mM of salt (see e.g., FIG. 1A). These droplets failed to coalesce for up to 2 h of incubation and formed clusters or chain-like assemblies, suggesting that these complexes are more viscous and have gel or solid-like properties. This is consistent with a previous report showing increased misfolded TDP-43 oligomeric species when the droplets were incubated for 2 h. It was then investigated whether association with A(GU)₆ RNA oligonucleotide (see e.g., TABLE 1), which strongly inhibits TDP-43 aggregation in vitro, alters the dynamic properties of TDP-43 droplets.

TABLE 1
Oligonucleotide sequences used in phase
separation assays.

| Name | RNA Oligonucleotide Sequence (5′→3′) |
|---|---|
| A(GU)₆ | AGUGUGUGUGUGU (SEQ ID NO: 4) |
| A(GU)₁₆ | AGUGUGUGUGUGUGUGUGUGUGUGUGUGUGUGUGUGU |
| | (SEQ ID NO: 1) |
| CLIP34 | GAGAGAGCGCGUGCAGAGACUUGGUGGUGCAUAA |
| | (SEQ ID NO: 2) |
| (AUG12)₃ | GUGUGAAUGAAUGUGUGAAUGAAUGUGUGAAUGAAU |
| | (SEQ ID NO: 3) |
| A(CA)₆ | ACACACACACACA (SEQ ID NO: 10) |
| NS | CGCGACGGACGGAAAGACCCCUAUCCGUCGCG |
| | (SEQ ID NO: 11) |
| Name | DNA Oligonucleotide Sequence (5′→3′) |
| A(GT)₁₈ | AGTGTGTGTGTGTGTGTGTGTGTGTGTGTGTGTGTGT |
| | (SEQ ID NO: 12) |
| NS ssDNA | CGCGACGGACGGAAAGACCCCUAUCCGUCGCG |
| | (SEQ ID NO: 13) |

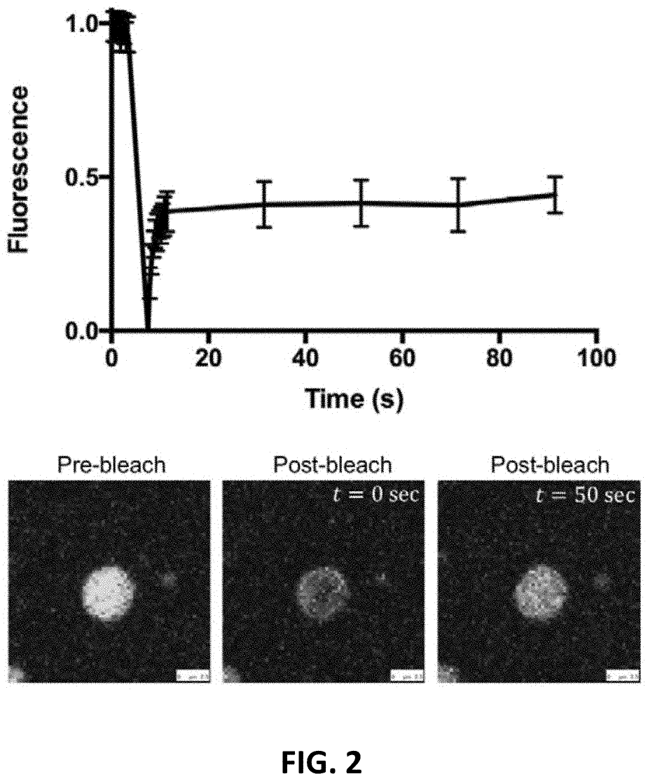

Addition of A(GU)₆ RNA dramatically increased the size of TDP-43 droplets compared to no RNA conditions (see e.g., FIG. 1B). The TDP-43 droplets in the presence of A(GU)₆ maintained liquid-like properties, as shown by fluorescence recovery after photobleaching (FRAP) (see e.g., FIG. 2). As additional control, an RNA oligonucleotide of similar length, A(CA)₆, which previously showed no significant binding activity for TDP-43 was used. In contrast with the GU-rich RNA molecule, the control RNA oligonucleotide did not promote TDP-43 droplet coalescence (see e.g., FIG. 1B). A(GU)₆ is predicted to interact with one TDP-43 molecule, based on biochemical and structural studies. Therefore, a longer RNA sequence of GU-repeats that potentially interacts with three TDP-43 molecules, A(GU)₁₅ (see e.g., TABLE 1) was used, and a dramatic increase in the size of TDP-43 droplets was observed compared to those formed in the presence of A(GU)₆ (see e.g., FIG. 1B). Together, these data indicate that RNA is a strong modulator of TDP-43 phase separation, promoting fusion and coalescence into large droplets, depending on sequence and length. In addition, these findings suggest that GU-rich oligonucleotides promote liquid-like behavior of TDP-43 condensates. This previously unexplored function of RNA may be linked to the ability of these RNA sequences to prevent TDP-43 misfolding.

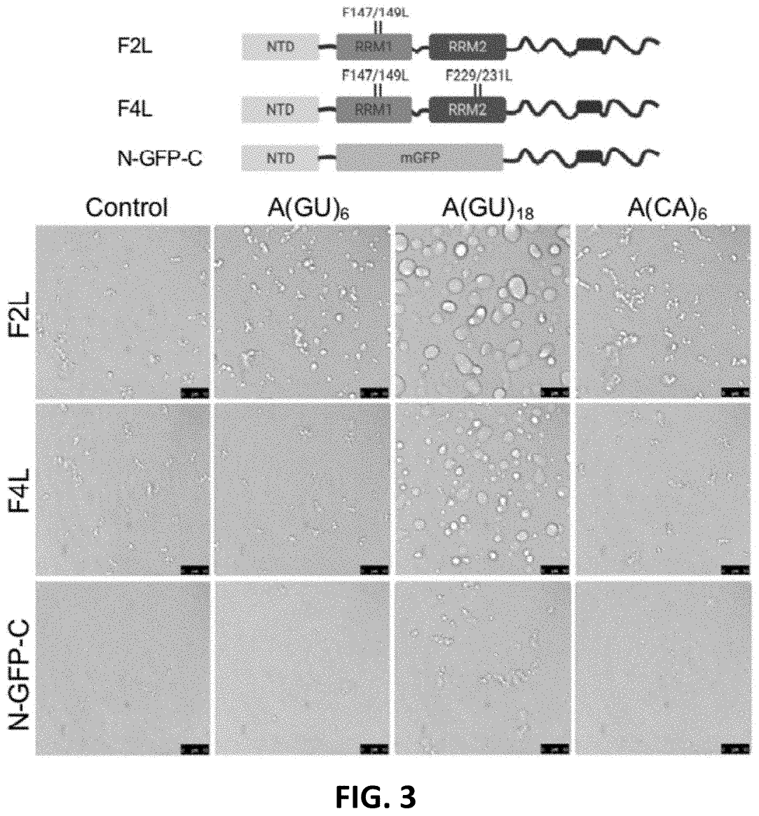

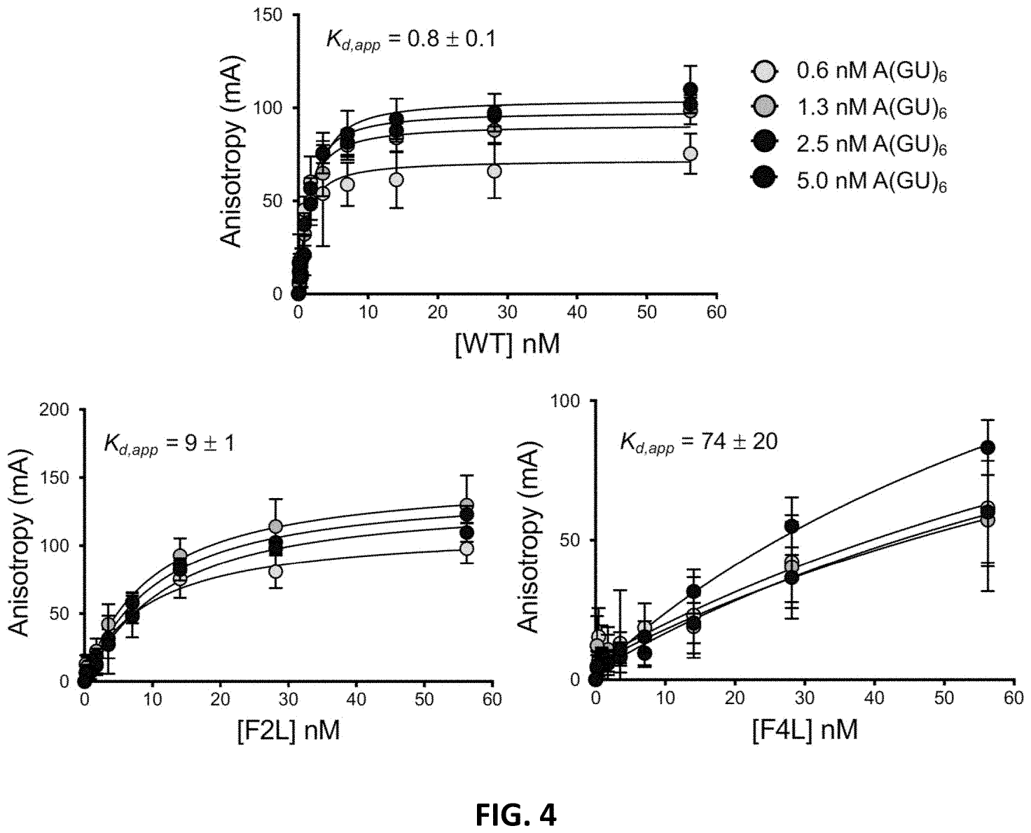

To further test the role of specific RNA binding in promoting dynamic, liquid properties of TDP-43-RNA droplets, three RNA binding-deficient TDP-43 mutants (see e.g., FIG. 3) were used. TDP-43 is composed of two tandem RNA recognition motifs (RRMs), which are important for specific binding to RNA and single-stranded DNA. Of the two RNA binding domains, RRM1 confers most of the binding affinity and sequence specificity to RNA. The mutations studied carry substitutions in RRM1 alone, F2L (Phe147/149Leu); mutations in RRM1 and RRM2, F4L (Phe147/149/229/231Leu); and a substitution of both RRMs with monomeric GFP (N-GFP-CTD). To confirm the expected loss in RNA-binding affinity of these mutants, the apparent dissociation constant (Kd,app) of wild-type (VVT) and mutants for GU-repeat RNA was measured by fluorescence anisotropy (see e.g., FIG. 4). These assays showed 11 and 92-fold loss in GU-rich RNA-binding affinity for F2L and F4L compared to WT, respectively (see e.g., TABLE 2).

TABLE 2
RNA-binding affinity for A(GU)₆ RNA.

| Protein | K_{d, app} (nM) | K^{Mut}_{d; app}/K^{WT}_{d; app}^{a} |
|---|---|---|
| Wild-type (WT) | 0.8 ± 0.1 | 1 |
| F2L (F147/149L) | 9 ± 1 | 11 |
| F4L (F147/149/F229/231L) | 74 ± 20 | 92 |
| ΔN (a.a. 102-414) | 0.7 ± 0.1 | 0.8 |
| Y4R/E17R | 1.7 ± 0.5 | 2 |
| RRM1-2 (a.a. 102-269) | 0.80 ± 0.16 | 1 |
| ΔC (a.a. 1-269) | 1.1 ± 0.2 | 1.4 |
| K181E | 3.6 ± 0.5 | 5 |
| A315E | 1.1 ± 0.3 | 1.4 |
| A315T | 0.6 ± 0.1 | 0.8 |
| A321G | 1.3 ± 0.2 | 1.6 |
| Q331K | 1.6 ± 0.3 | 2 |
| M337V | 2.4 ± 0.5 | 3 |
| A382T | 0.7 ± 0.2 | 0.9 |

^{a}Estimated loss (>1) or gain (<1) in binding affinity relative to WT.

Binding in the case of N-GFP-CTD was not measured. In the absence of RNA, F2L and F4L formed small droplets that were not significantly different from WT droplets shown in FIG. 1B (see e.g., FIG. 3). However, in the presence of A(GU)₆ RNA, F2L formed smaller droplets and decreased coalescence, compared to WT, that were not different from those in the presence of A(CA)₆ RNA. Furthermore, addition of A(GU)₆ did not significantly alter the behavior of F4L and N-GFP-CTD, compared to no RNA and A(CA)₆ control, forming small condensates and fibrillar structures. Addition of the longer A(GU)₁₈ RNA rescued phase separation in the case of F2L and partially recovered it in the case of F4L (see e.g., FIG. 3). In contrast, complete substitution of the RRMs in the NGFP-CTD chimera abolished the ability of RNA to modulate condensation even in the presence of A(GU)₁₈. Collectively, these results suggest that disrupting RNA-TDP-43 interactions in the RRM1/2 region greatly impairs the ability of RNA composed of a single binding module, A(GU)₆, to promote droplet liquid-like properties. However, in the presence of RNA with multiple binding sites, which bring multiple TDP-43 molecules in close proximity, LLPS of F2L and F4L is rescued and partially recovered, respectively. This may be explained by the data showing that these mutations substantially lower the affinity of TDP-43 for the chosen GU-rich RNA sequences, but still maintain nanomolar affinities for these sequences (see e.g., TABLE 2), perhaps explaining the incomplete inhibition of A(GU)₁₈-driven LLPS. This process is no longer possible if specific RNA binding through the RRMs is entirely absent, as shown in the case of N-GFP-C, where the RRM1-2 region is substituted by mGFP. The observations with the mGFP chimera also suggest that the N and C-terminal TDP-43 domains play a minimal role in GU-driven LLPS in the absence of the RRMs because of the lack of liquid droplets in any of the conditions (see e.g., FIG. 3).

RNA Binding to Multiple Sites Strongly Induces TDP-43 Phase Separation

Based on the results discussed above, how RNA sequences that recruit multiple TDP-43 molecules affect phase separation was investigated. In particular, surveying RNA molecules representative of natural TDP-43 targets, including GU repeat tracks was investigated. It was previously shown that a minimum of five GU repeats binds one molecule of TDP-43 and that GU repeats may be interrupted by nonspecific residues without a significant loss in binding affinity. This was confirmed by structural studies in which binding of one TDP-43 molecule covers approximately 10-12 nucleotides through specific interactions with the RRMs. Therefore, the natural TDP-43-bound RNA sequences, consisting of long stretches of GU-rich sequences (GU-rich clusters), indicate extended regions of multiple TDP-43-binding sites.

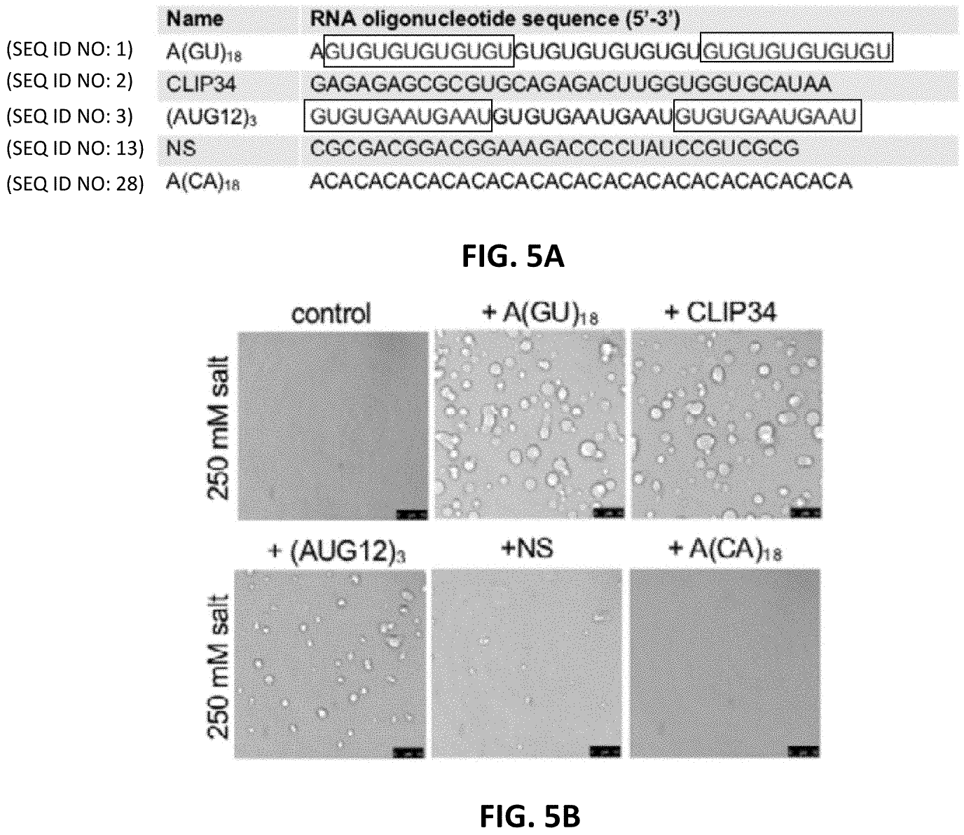

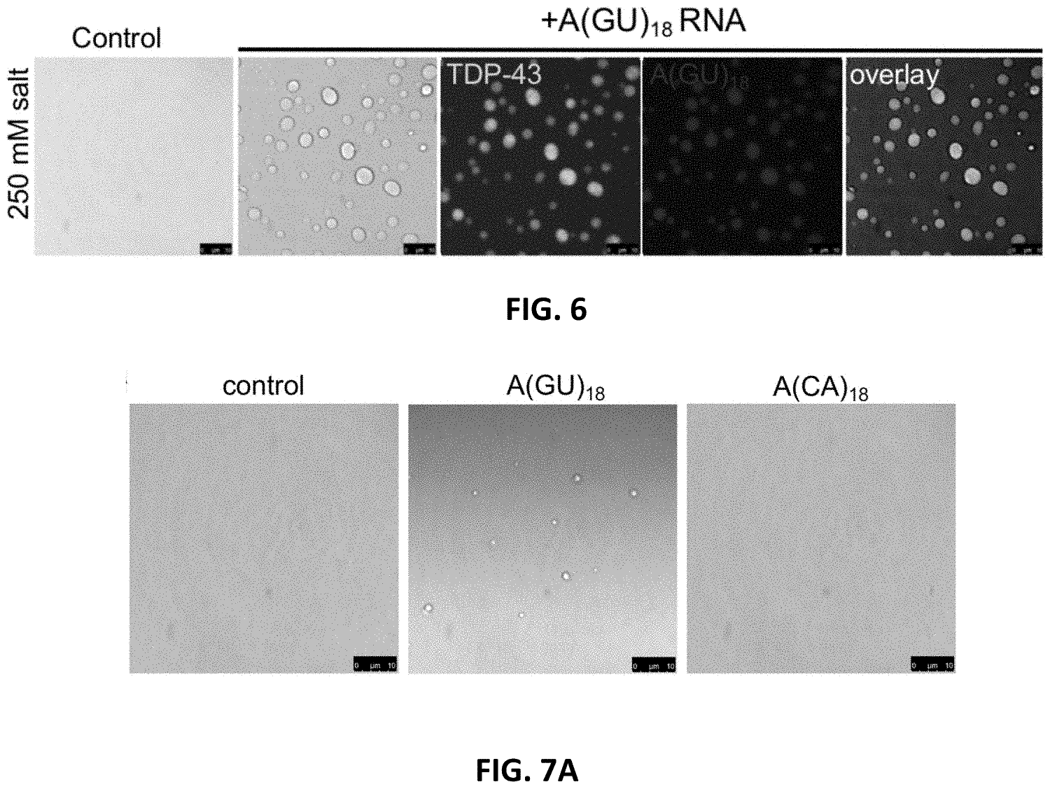

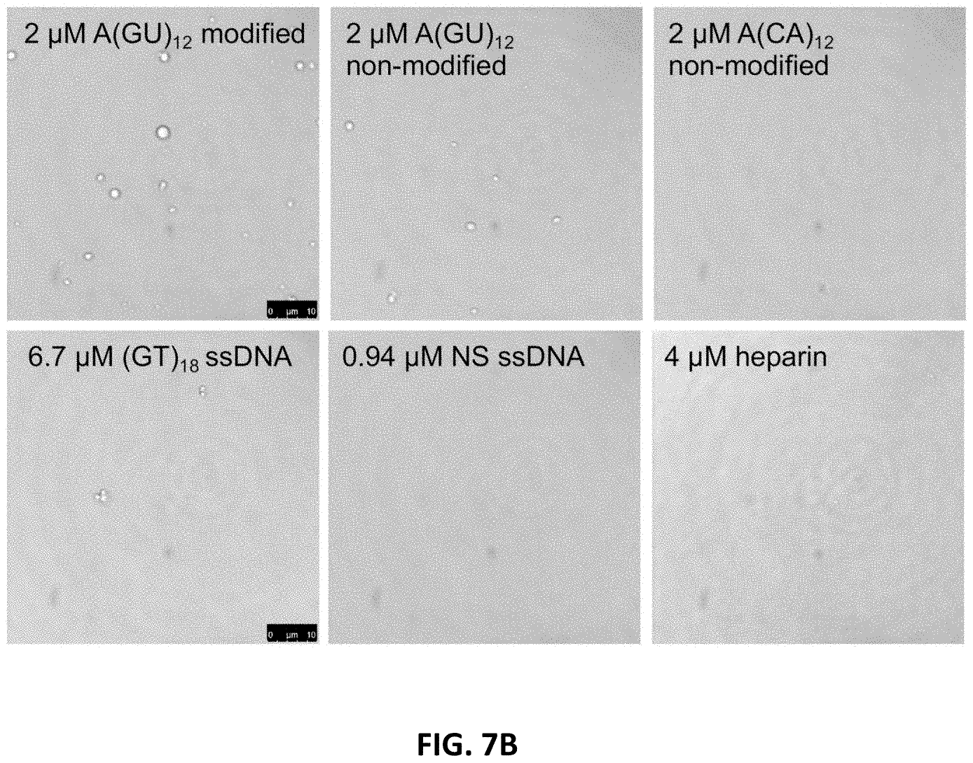

Examples of these sequences include a GT repeat polymorphism (9-15 repeats) in the cystic fibrosis transmembrane conductance regulator (CFTR) modulating exon 9 splicing and GT-repeat stretches (10 up to 40 repeats) found in introns and 3′ UTR of Cdk6. To explore how RNA molecules similarly composed of extended GU-rich sequences affect LLPS properties of TDP-43, droplet formation at higher salt (250 mM of NaCl) was studied. Under these conditions, TDP-43 did not phase separate on its own (see e.g., FIG. 5A-FIG. 5B), however, addition of A(GU)₁₈ led to the spontaneous formation of protein droplets, which entirely colocalized with RNA (see e.g., FIG. 6). The dynamic behavior of the A(GU)₁₈-mediated TDP-43 droplets was investigated to determine whether these were consistent with LLPS properties. Spherical condensates that fused and coalesced into larger droplets were reproducibly observed. In addition, increasing salt concentration to the preformed droplets dissolved the dense phase, indicating the reversibility of the multicomponent complex. The next set of experiments was conducted to test the specificity of GU-rich RNA molecules in modulating TDP-43 phase separation. To facilitate other experiments using purified full-length TDP-43, which is highly aggregation-prone, the protein was N-terminally tagged with the yeast SUMO ortholog Smt3. Therefore, it was important to evaluate whether the tag influenced TDP-43 condensation driven by GU-rich RNA by removing SUMO, as discussed below. FIG. 7A shows that A(GU)₁₈ RNA triggered condensation of untagged TDP-43 at 250 mM of NaCl, while no LLPS was observed upon mixing with A(CA)₁₈, or in control in the absence of RNA. The amount of protein recovered following SUMO cleavage and isolation of TDP-43 was considerably less than that used for the experiments with tagged TDP-43, estimated at 0.2-0.5 μM vs. 4 μM, respectively. These lower concentrations were probably caused by a drastic loss of soluble protein upon removal of the N-terminal SUMO. The differences in concentration may explain the smaller sized condensates observed for the A(GU)₁₈-induced condensates formed by the untagged protein relative to SUMO-tagged TDP-43 (see e.g., FIG. 5B). Nevertheless, while the final yield of the untagged protein was low, untagged TDP-43 droplets were generated only in the presence of long GU-repeat RNA. The effect of charged polymers other than long GU-rich RNA oligonucleotides on TDP-43 condensation was also determined. Heparin or a non-specific single-stranded DNA (ssDNA) oligonucleotide did not affect TDP-43 phase separation under the conditions tested (see e.g., FIG. 7B). ssDNA composed of GT repeats A(GT)₁₈, which binds TDP-43 through RRM interactions with approximately 200-fold lower affinity than the analogous RNA sequence, did not induce TDP-43 droplet formation at concentrations that strongly promoted phase separation in the case of A (GU)₁₈ (see e.g., FIG. 7B). For these studies, modified RNA oligonucleotides were used to increase stability. The modification did not alter the ability of RNA to drive TDP-43 LLPS under the conditions tested, as seen by using analogous nonmodified RNA (see e.g., FIG. 7B).

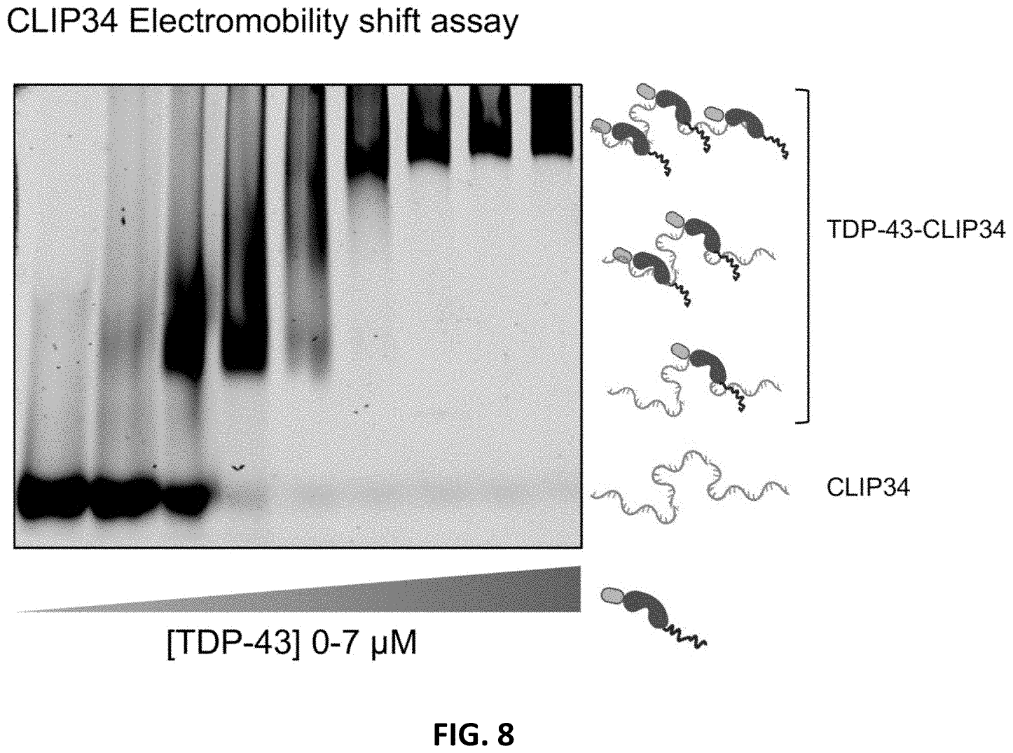

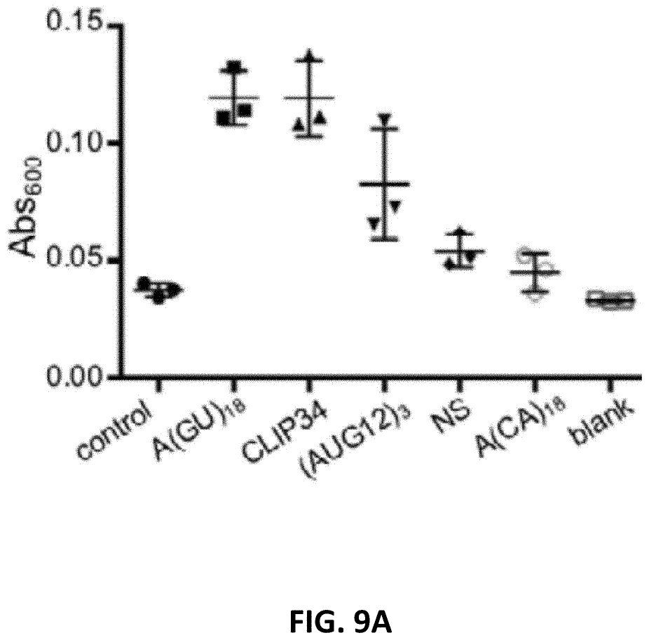

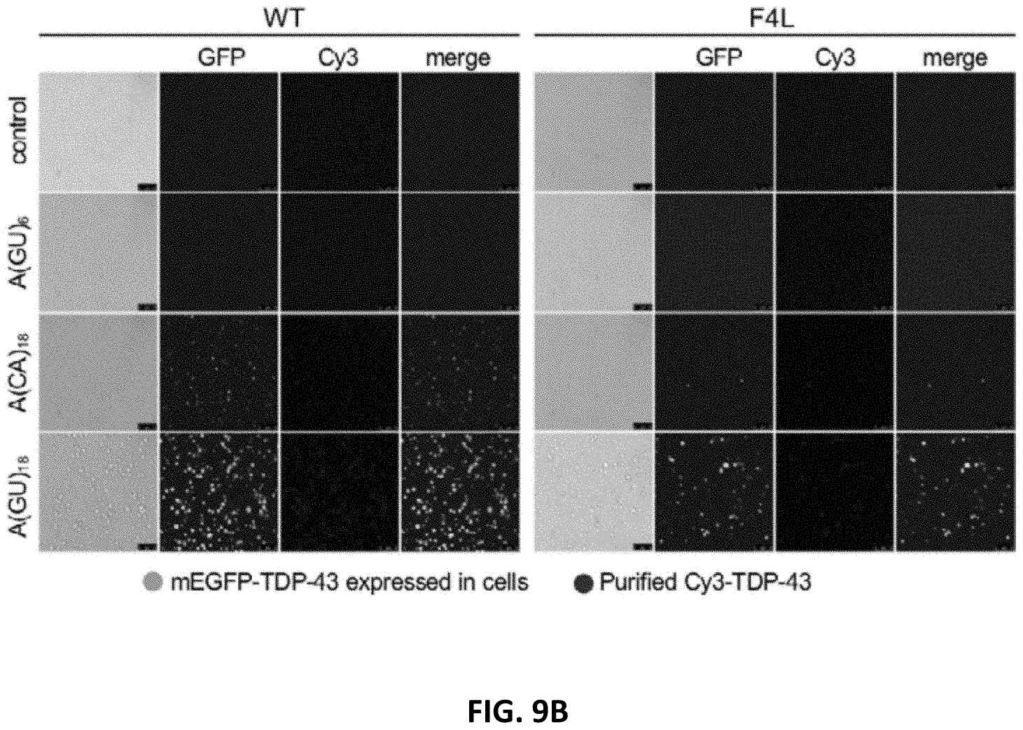

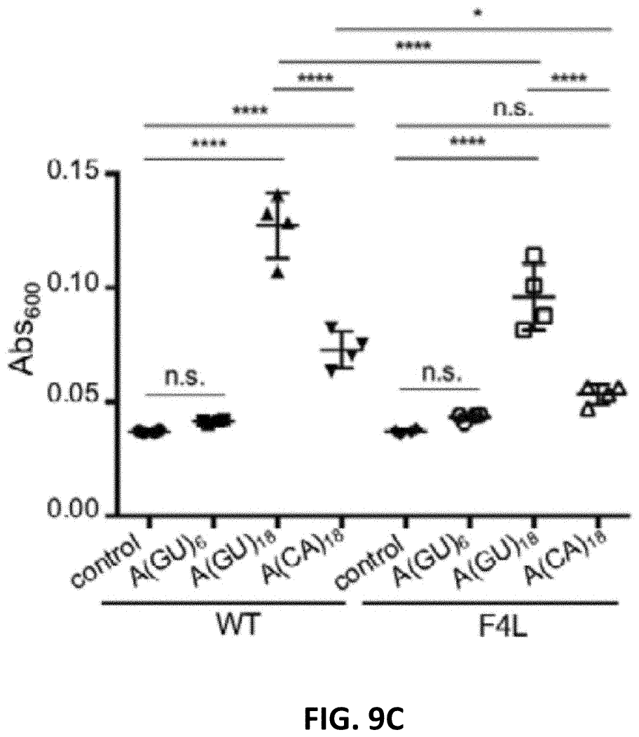

Next, the regulation of TDP-43 LLPS by alternative RNA sequences that may potentially bind multiple protein molecules, but are not composed of GU-repeats (see e.g., FIG. 5A), was analyzed. These sequences are equivalent to those found in physiological targets. The consensus sequence found among natural TDP-43-bound RNA transcripts in human cells, AUG12 binds one molecule of TDP-43. An RNA oligonucleotide composed of AUG12 repeated three times, (AUG12)₃, was generated. In addition, the effect of CLIP34, a 34-nucleotide sequence derived from the Tardbp 3′ UTR that recruits TDP-43 during autoregulation was analyzed. This sequence differs from GU repeat RNA as it is not particularly abundant in GU motifs. The CLIP34 oligonucleotide used herein bound multiple TDP-43 molecules, accommodating at least three as estimated by electromobility shift assays (EMSA) (see e.g., FIG. 8). At 250 mM of salt, CLIP34 triggered spontaneous TDP-43 phase separation into large droplets, similar to A(GU)₁₈ (see e.g., FIG. 5B). Addition of (AUG12)₃ also induced droplets, although these were smaller compared to those driven by A(GU)₁₈ and CLIP34 at similar RNA concentrations (see e.g., FIG. 5B). In contrast, an alternative RNA oligonucleotide of similar length, NS (32 nucleotides), consistently showed few small condensates mixed with fibrillar structures (see e.g., FIG. 5B). Additionally, the sequence A(CA)₁₈ showed no effect on TDP-43 LLPS. Furthermore, turbidity measurements taken as absorbance at 600 nm showed readings consistent with what was observed microscopically (see e.g., FIG. 9A). Addition of A(GU)₁₈ and CLIP34 generated the greatest increase in turbidity while the nonspecific sequences NS and A(CA)₁₈ showed values closer to control in the absence of RNA. In all, these observations provide strong evidence that RNA drives the formation of TDP-43 condensates exhibiting liquid-like properties. In addition, the ability to promote TDP-43 demixing is unique to RNA sequences that are established to interact with TDP-43 specifically and may bind to more than one TDP-43 molecule. These RNA oligonucleotides bind TDP-43 with high affinity in vitro and are composed of sequences commonly found among physiological targets.

Long GU-Repeat RNA Promotes LLPS of TDP-43 in Mammalian Cell Lysate

The assays described herein had tested whether RNA could induce TDP-43 LLPS in a simplified system combining primarily purified protein and RNA. However, the cellular milieu is much more complex. Whether RNA-driven TDP-43 condensates could be reconstituted in an environment composed of cellular cytoplasmic and nuclear components was investigated. To this end, a new protocol was established to analyze cellular extract from HEK293 cells that stably express a single copy of either mEGFP-TDP-43 WT or the RNA binding-deficient mutant mEGFP-TDP-43 F4L. The methods were adapted from recent studies that reconstitute stress granules and nucleoli in cellular lysates. Cellular lysate was prepared after inducing the expression of GFP-tagged TDP-43 and Cy3-labeled recombinant wild-type TDP-43 was added. In the absence of RNA, the mixture appeared homogenous and lacked visible foci (see e.g., FIG. 9B). Likewise, when A(GU)₆ RNA, which favors binding one TDP-43 molecule, was added, the mixture remained free of liquid droplets and showed no significant increase in turbidity compared to control (see e.g., FIG. 9B-FIG. 9C). However, when A(GU)₁₈, which consists of three potential TDP-43-binding sites, was added, spherical droplets containing both mEGFP-labeled cellular and Cy3-labeled recombinant TDP-43 formed (see e.g., FIG. 9B). Consistently, addition of A(GU)₁₈ led to an almost 4-fold increase in turbidity compared to control or in the presence of A(GU)₆ RNA (see e.g., FIG. 9C). Unlike in the LLPS assay in the absence of cellular lysate, A(CA)₁₈ RNA also generated a number of droplets, albeit far fewer than A(GU)₁₈. A corresponding significant increase in turbidity was also seen upon mixing with A(CA)₁₈ RNA (see e.g., FIG. 9C), however this was 50% lower than in the presence of A(GU)₁₈. The effect of A(CA)₁₈ may be due to the increased concentration of both TDP-43 and RNA in this assay, or to the contribution of other RNA-binding proteins present in the cell lysate. Lysate from cells expressing mEGFP-TDP-43 F4L combined with purified Cy3-labeled TDP-43 F4L in the presence of A (GU)₁₈ showed a readily noticeable decrease in the number of droplets, compared to WT (see e.g., FIG. 9B). Correspondingly, the TDP-43 F4L lysate showed decreased turbidity relative to WT TDP-43 lysate (see e.g., FIG. 9C) under phase separation-promoting conditions. In agreement with the observations using purified protein and RNA, these experiments show that specific TDP-43 interactions with RNA composed of multiple binding sites induces LLPS in a cellular environment. Furthermore, this new technique may be used to reconstitute TDP-43 RNP granules and interrogate their regulation in the presence of other cellular components.

RNA-Driven TDP-43 Phase Separation is Sequence-Dependent and is Modulated by the Number of Binding Sites

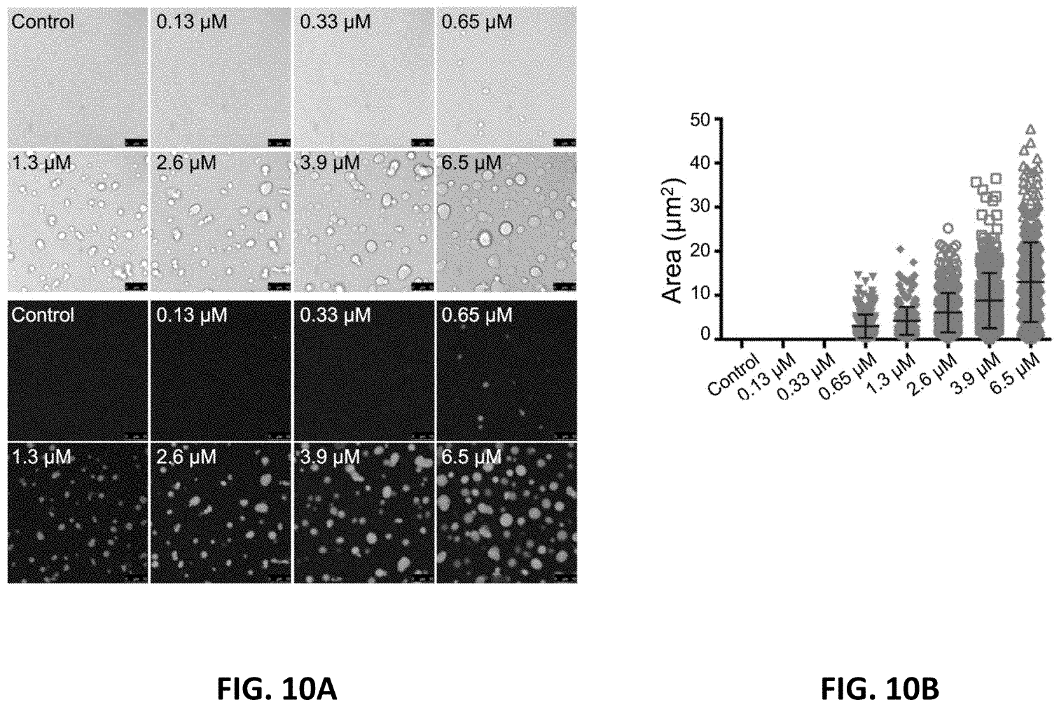

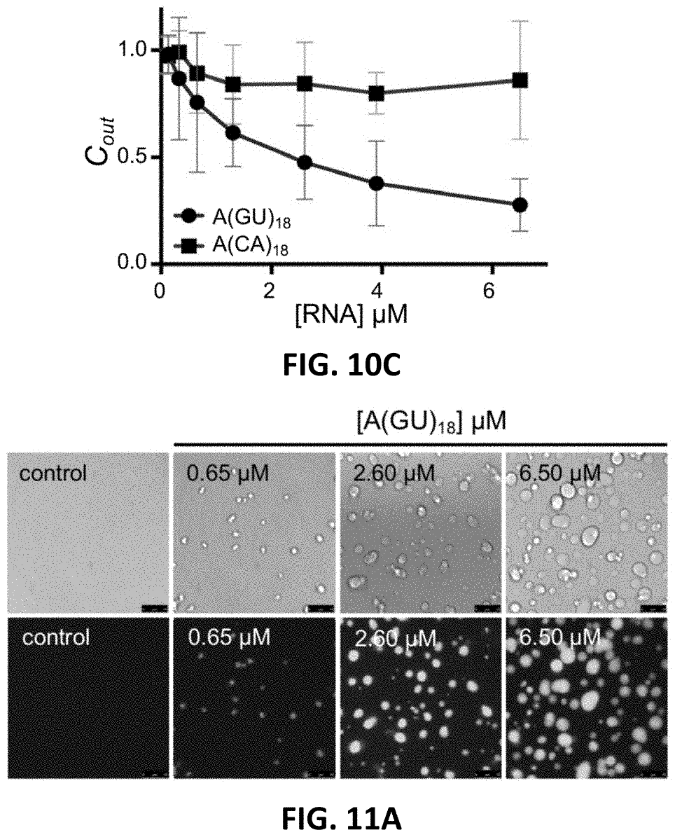

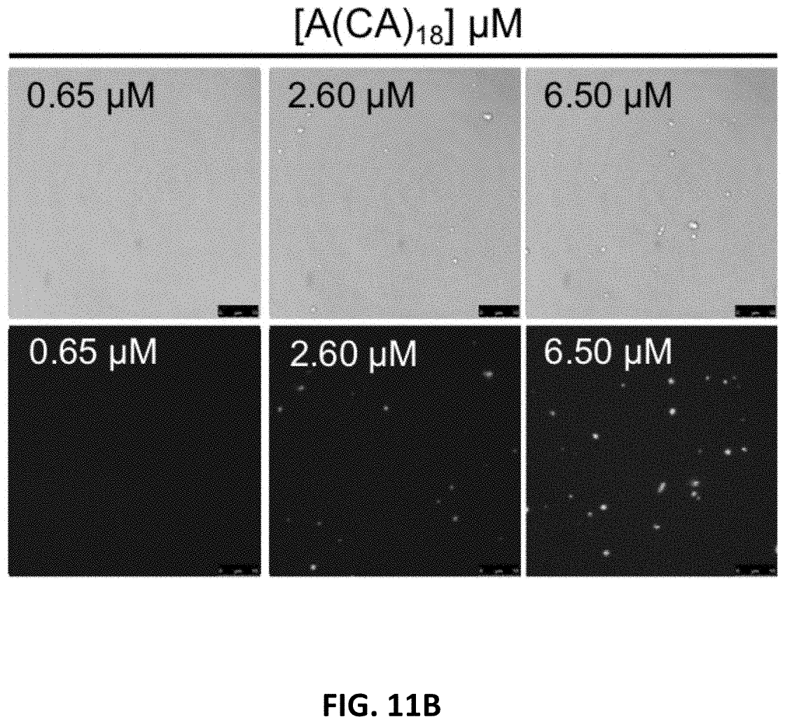

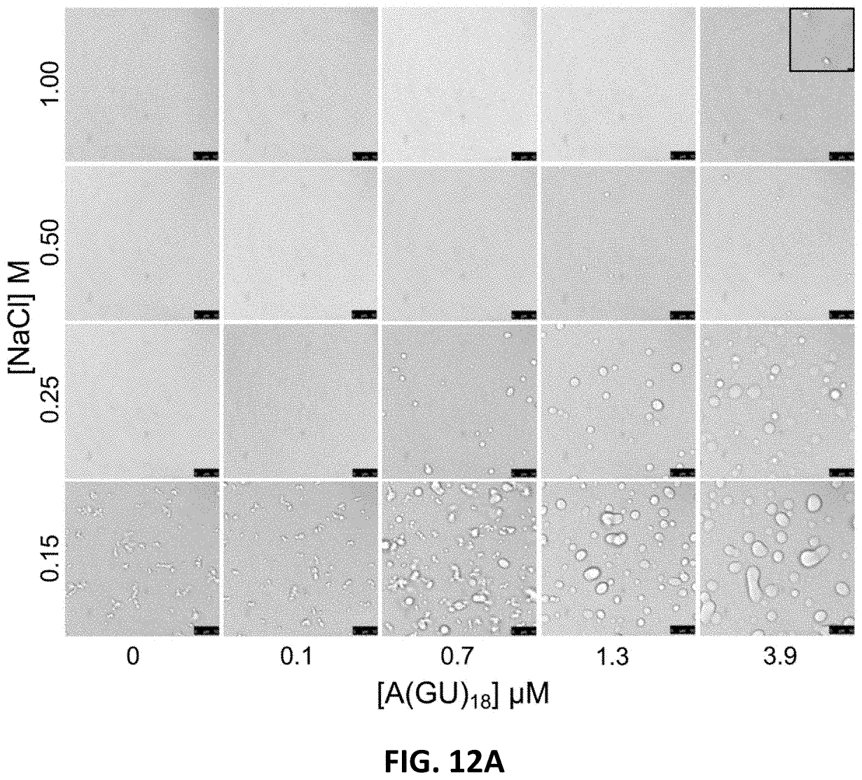

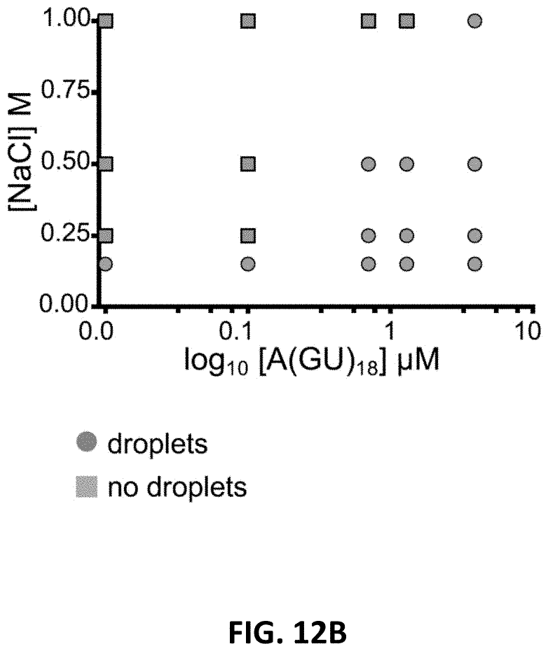

Next, the role of GU-rich RNA as a modulator of TDP-43 phase properties was further investigated. First, it was observed that TDP-43 droplets grew larger as A(GU)₁₈ concentration increased (quantified by droplet area), suggesting that the volume of the dense phase increased as a function of RNA (see e.g., FIG. 10A-FIG. 10B). The concentration of TDP-43 in the light phase (Cₒᵤₜ) was also quantified by Bradford protein analysis following separation of dense and light phases. As the size of the droplets grew with increasing A(GU)₁₈ concentration, Cₒᵤₜ decreased (see e.g., FIG. 10C). The highest A(GU)₁₈ concentration led to approximately 70% reduction of TDP-43 concentration in the light phase relative to control, in the absence of RNA. Additionally, the effect of increasing concentrations of A(CA)₁₈ on TDP-43 LLPS was tested and substantially fewer and smaller condensates were observed than were present in corresponding A(GU)₁₈ conditions (see e.g., FIG. 11A-FIG. 11B). In accordance with this data, Cₒᵤₜ of A(CA)₁₈ samples showed substantially less change from baseline than did A(GU)₁₈, suggesting less TDP-43 accumulation in condensates that do form under nonspecific RNA conditions (see e.g., FIG. 10C). These data strongly indicate that TDP-43 accumulation in the dense phase is upregulated as a function of GU-rich RNA presence specifically. To explore whether A(GU)₁₈ altered the TDP-43 phase boundary, droplet formation was analyzed at different salt concentrations as a function of A(GU)₁₈ concentration. At equal TDP-43 concentration, the increase in salt inhibited droplet formation regardless of RNA presence (see e.g., FIG. 12A). However, A(GU)₁₈ promoted droplet formation at salt concentrations that were not permissive in the absence of RNA. A schematic representation of droplet formation as a function of salt indicates a shift in the TDP-43 phase boundary driven by RNA (see e.g., FIG. 12B), suggesting a decrease in the saturation concentration (Cₛₐₜ) of TDP-43 necessary to initiate condensation. These results are consistent with the effect of GU-rich RNA on driving TDP-43 LLPS and provide strong evidence that RNA composed of multiple binding sites modifies TDP-43 phase behavior by favoring condensation.

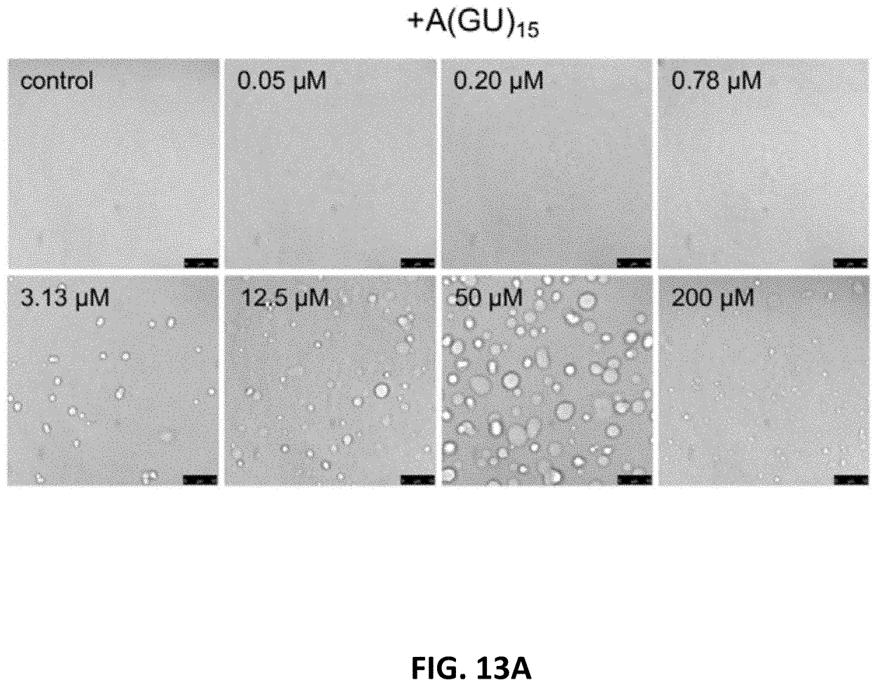

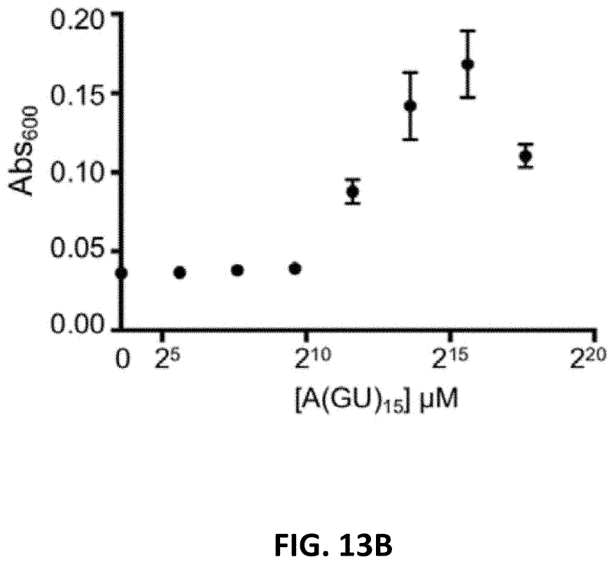

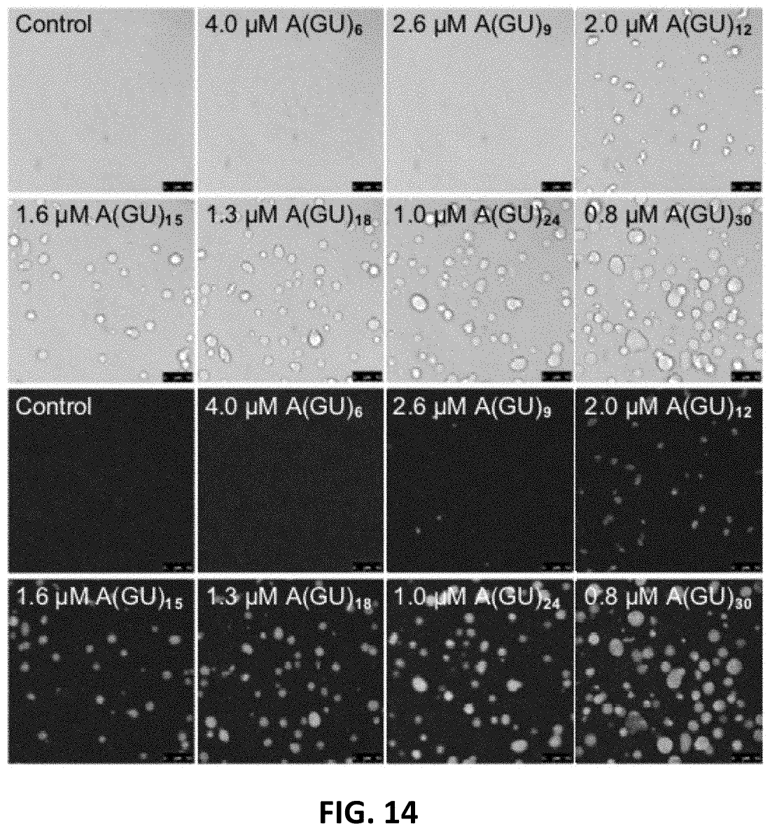

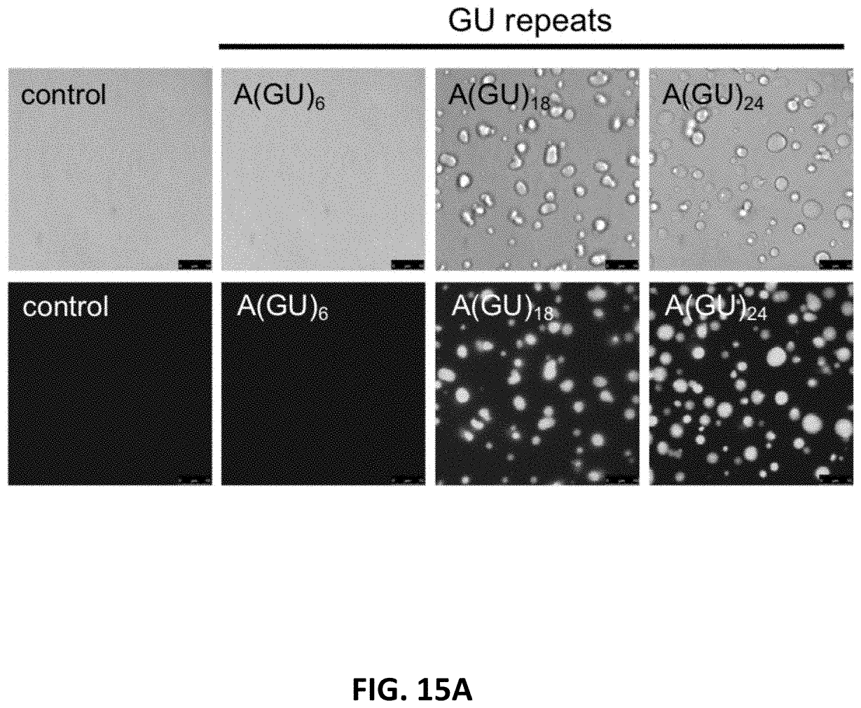

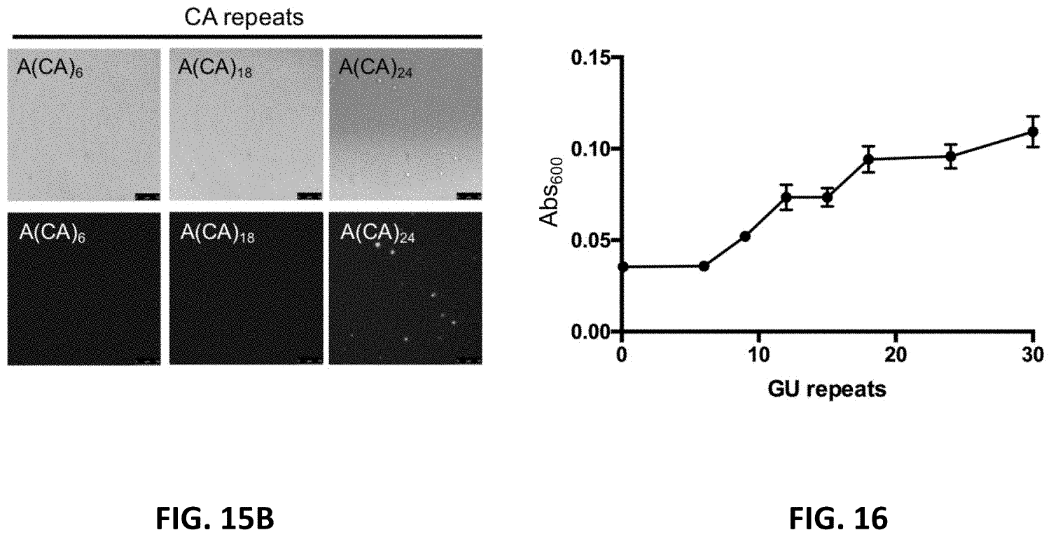

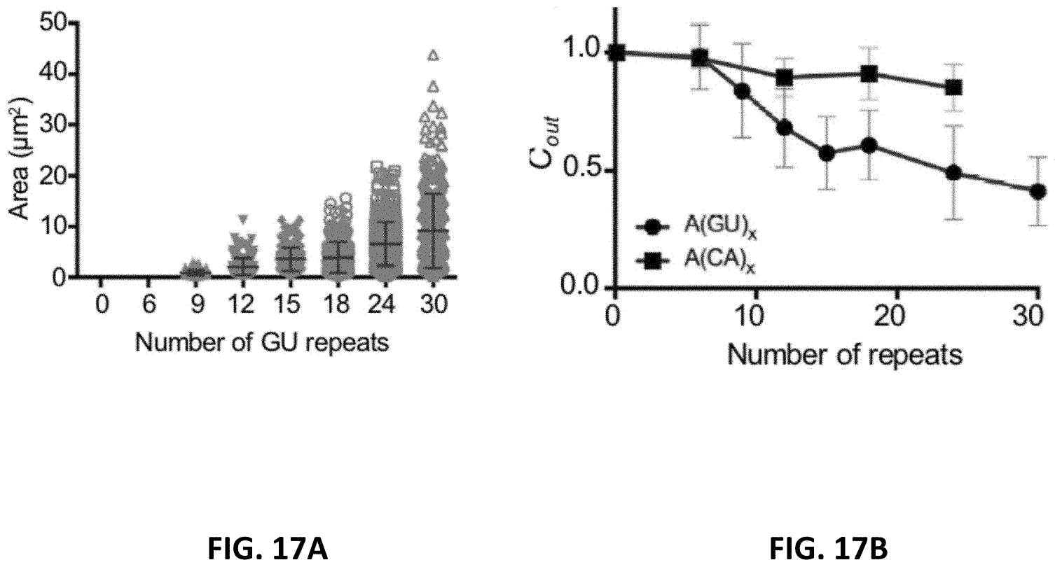

Previous studies showed that at high concentrations, nonspecific RNA drives TDP-43 away from phase separation, thus it was investigated whether high concentrations of specific RNA would have the same effect. Using A(GU)₁₅, the concentration of RNA was increased while keeping the TDP-43 concentration constant, up to a 50-fold molar excess of RNA. TDP-43 liquid droplets and turbidity continued to increase up to 50 μM A(GU)₁₅ and sharply declined at the highest RNA concentration (see e.g., FIG. 13A-FIG. 13B). These results support the idea that at very high concentrations, RNA may act as a suppressor of TDP-43 phase separation, as previously suggested. It is also possible that at the concentrations generally used in these studies, long GU repeat RNA recruits multiple TDP-43 molecules, driving phase separation. However, LLPS may be reversed at very high RNA concentrations, where excess RNA results in binding one TDP-43 per RNA molecule. While this is a possible scenario, reentry into a single-phase system only occurred at the highest A(GU)₁₅ concentration tested, and this may be due to the preference of these RNA oligonucleotides to bind multiple TDP-43 molecules. This is based on the binding cooperativity of TDP-43 upon association with long GU repeat RNA as previously reported. Next, it was investigated whether increasing multivalent association of TDP-43 molecules on an RNA template increases protein condensation. For these experiments, LLPS was studied as a function of the number of contiguous potential TDP-43-binding sites. The concentration of GU- or CA-rich RNA oligonucleotides was modified for each sequence to maintain the number of TDP-43-binding sites constant across samples. In doing so, the absolute amount of RNA did not change from one condition to the next, rather the only change was the number of potential TDP-43-binding sites that were linked together. At 250 mM of NaCl, addition of A(GU)₆ RNA, which favors binding of one TDP-43 molecule, did not promote TDP-43 liquid droplet formation (see e.g., FIG. 14). Small condensates started to form in the presence of A(GU)₉ and these grew larger as a function of GU-repeat length, while condensates were not visible microscopically with CA-rich sequences until a repeat number of 24 (see e.g., FIG. 15A-FIG. 15B). TDP-43 LLPS was also analyzed by measuring turbidity in the presence of the different GU-repeat RNA oligonucleotides (see e.g., FIG. 16). In agreement with the imaging data, starting with A(GU)₉, turbidity increased as the length of GU repeats in the sample increased. Further quantification of TDP-43 condensation triggered by increasing GU-repeat length, showed that droplet area directly increased with the number of TDP-43-binding sites (see e.g., FIG. 17A). In addition, TDP-43 concentration in the light phase (Cₒᵤₜ) decreased proportionally with greater GU-repeat number (see e.g., FIG. 17B), suggesting a corresponding increase of TDP-43 in the dense phase as the number of GU-repeats increased. In stark contrast, TDP-43 Cₒᵤₜ only moderately decreased with increasing CA repeat number, compared to control in the absence of RNA (see e.g., FIG. 17B). Collectively, the observations indicate that TDP-43 condensation is strongly regulated by number of proximal RNA-binding sites. These findings open avenues to investigate how different TDP-43 RNA targets, which are composed of varying numbers of binding modules and varying binding affinity, may differentially regulate protein phase separation.

RNA-Driven TDP-43 Phase Separation is Mediated by N-Terminal and Low Complexity Domain Interactions

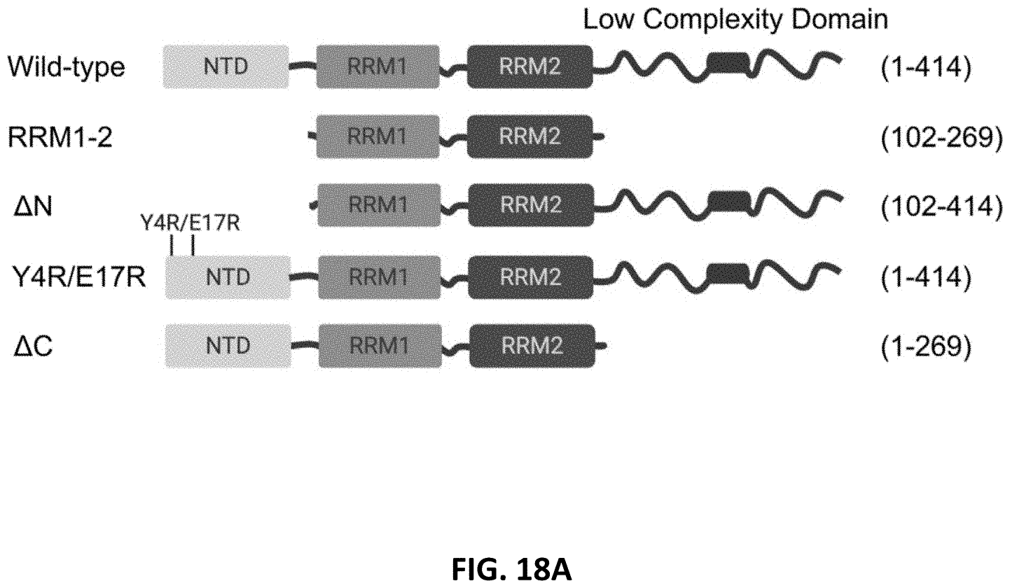

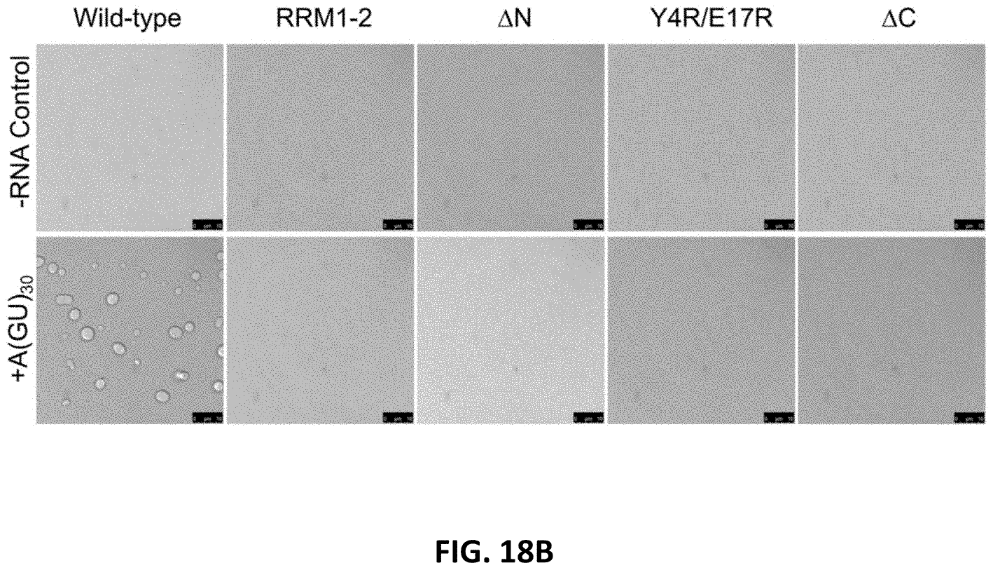

To further investigate the mechanism of TDP-43 RNA-driven phase separation, whether this process is also affected by TDP-43 domains that participate in protein-protein interactions and self-assembly was investigated. TDP-43 is organized into three independently folded domains followed by a mostly disordered region at the carboxyl terminus (see e.g., FIG. 18A). These four domains all contribute to TDP-43 self-assembly and RNA processing function. The N-terminal domain (NTD) folds into a homeobox-like structure that promotes TDP-43 oligomerization. NTD-driven complexes are necessary for protein function in RNA processing, phase separation, and for preventing the accumulation of RNA:DNA hybrid. This region is followed by the two RRMs and the carboxy-terminal domain (CTD), which is referred to as low complexity or prion-like domain for its similarity to yeast prion domains.

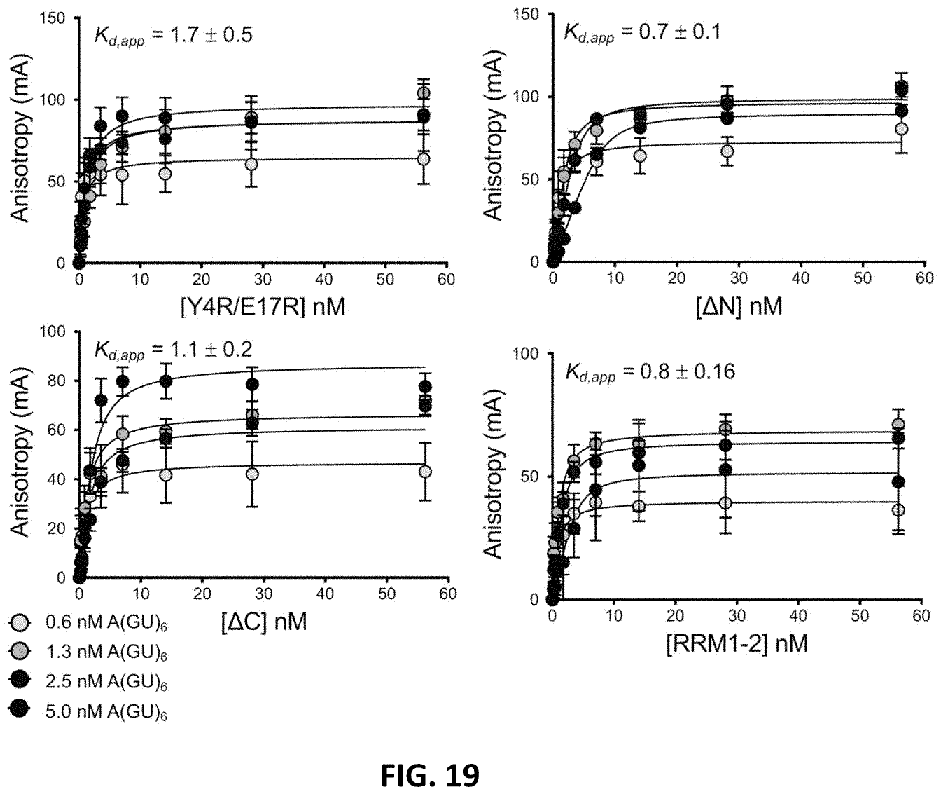

The CTD is a mostly disordered region that undergoes phase separation alone, suggesting that it plays an important role in full-length TDP-43 condensation. At the same time, this domain is a primary driver of protein aggregation and is also where almost all ALS and FTD-linked mutations are found. Droplet formation was investigated using WT, mutant, and TDP-43 fragments targeting the NTD and CTD (see e.g., FIG. 18A) at 250 mM of salt. In the absence of RNA, none of the constructs showed phase separation (see e.g., FIG. 18B). Deletion of both NTD and CTD (RRM1-2) precluded TDP-43 phase separation in the presence of A(GU)₃₀, in contrast to WT (see e.g., FIG. 18B). A fragment lacking the entire NTD (ΔN) or two site substitutions that prevent NTD-driven oligomerization (Y4R/E17R) completely blocked TDP-43 phase separation in the presence of RNA (see e.g., FIG. 18B). Similarly, RNA did not promote TDP-43 LLPS in the absence of the low complexity domain (see e.g., FIG. 18B). The K_{d,app} of WT and all the mutants for GU-repeat RNA (see e.g., FIG. 19 and TABLE 2) was measured and values were consistent with those previously measured for full-length WT and fragments, including AC and RRM1-2, using other methods. In all cases, the mutations did not significantly alter the binding affinity for GU-repeats, relative to WT. This suggests that the lack of A(GU)₃₀-induced droplet formation by the mutants is unlikely to be caused by reduced RNA interactions. Instead, the data indicate that binding of RRM1 and RRM2 to RNA is essential, but under the conditions tested is not sufficient to promote RNA-driven TDP-43 LLPS. The N-terminal domain oligomerization as well as CTD interactions play a major role in the RNA-mediated process, which is consistent with the importance of multivalent interactions in biomolecular condensation.

ALS-Linked Mutations Compromise RNA-Driven Liquid-Like Behavior of TDP-43 Condensates

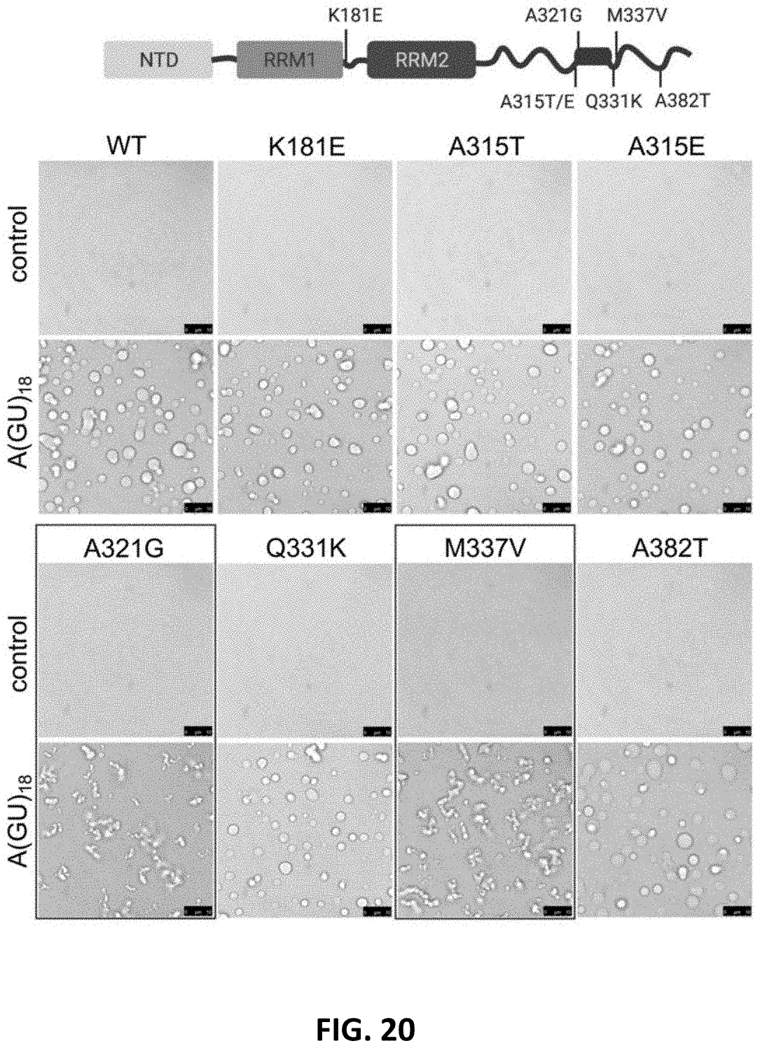

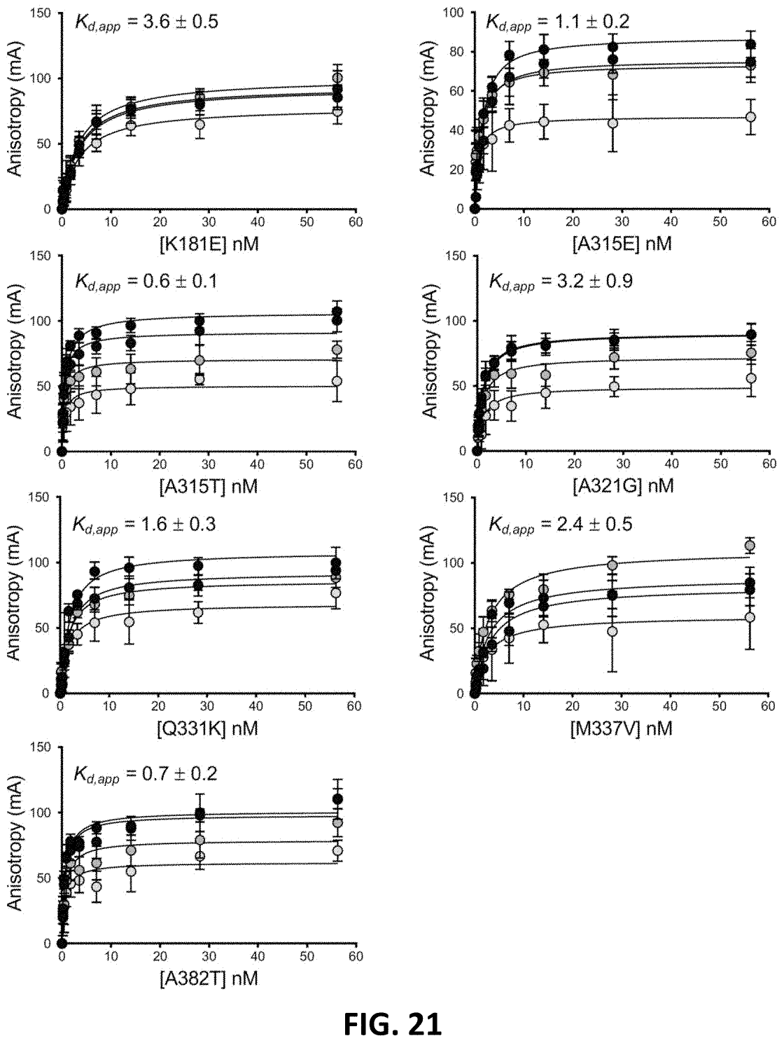

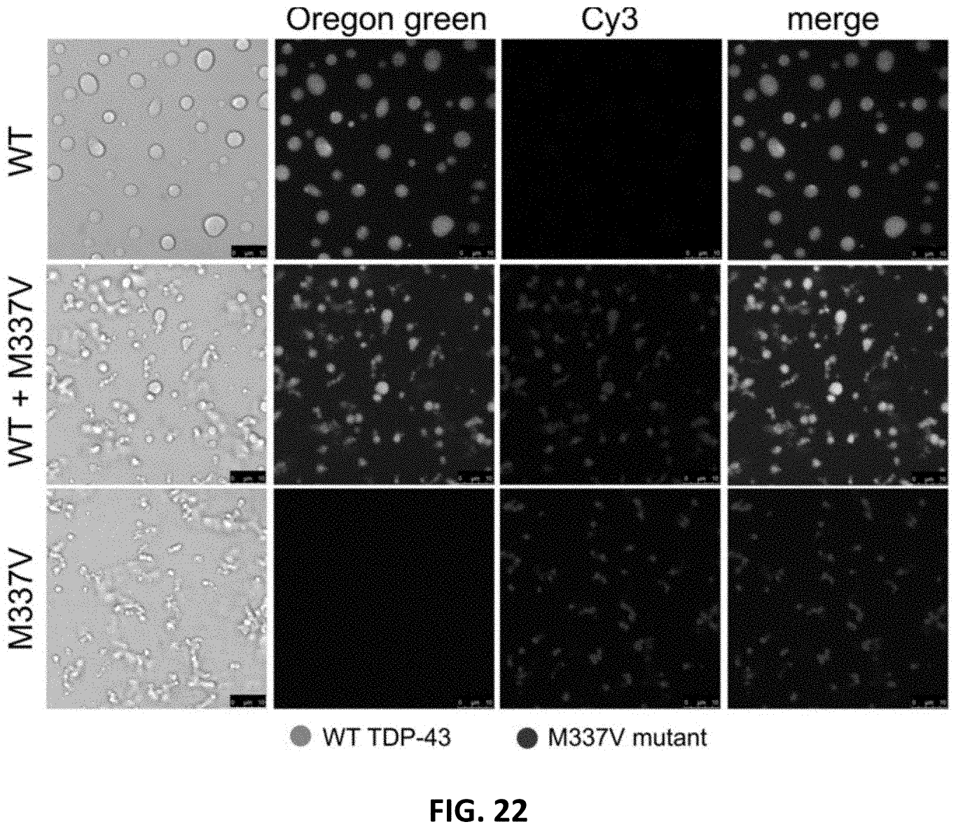

TDP-43 phase separation has been the focus of much investigation because disruption of LLPS homeostasis may underlie pathogenesis linked to protein aggregation and loss of function in neurodegeneration. To shed light on disease mechanisms the behavior of TDP-43 mutations causative of ALS/FTD was analyzed. A group of these, including K181E, A315E, A315T, A321G, Q331K, M337V, and A382T, was selected to determine whether they altered RNA-driven TDP-43 condensation. The recently identified K181E mutation is positioned in the linker region between RRM1 and RRM2 and was shown to increase aggregation in cells. All the other substitutions are in the vicinity or within an evolutionarily conserved region of the CTD (a.a. 320-343) which forms an alphahelical structure (a.a. 321-330, boxed region in FIG. 20) that extends further (up to a.a. 340) upon interaction with the same region of a second CTD molecule. This structure and the helix-helix association is central for TDP-43 LLPS and previous studies showed that these ALS mutations disrupt the intermolecular CTD interactions, drastically reducing LLPS. In addition, it was previously shown that some of these mutants, e.g., M337V and A315T, accelerate TDP-43 aggregation and increase intracellular aggregate seeding. RNA-driven LLPS of WT and the mutants was compared in control conditions without RNA or in the presence of A(GU)₁₈ RNA at 250 mM of salt. The microscopy analyses showed no significant changes in the RNA-driven LLPS of K181E, A315E, A315T, Q331K, and A382T relative to WT (see e.g., FIG. 20). In contrast, A321G and M337V strongly changed the phase separation dynamics in the presence of A(GU)₁₈ (see e.g., FIG. 20). The A321G and M337V condensates induced by the addition of A(GU)₁₈ were noticeably smaller than those formed by WT and assembled in clusters without fusing into larger droplets. This is highlighted observations of M337V droplets over time showing a stark contrast to WT behavior. The binding affinity was measured for GU-rich RNA of all the mutations tested and no significant changes relative to WT were found (see e.g., FIG. 21 and TABLE 2). Therefore, the results suggest that impaired liquid properties of the RNA-mediated M337V and A321G condensates are not caused by defects in RNA association. In addition, whether M337V condensates could interact with WT and impact the liquid properties of WT RNA-mediated assemblies was tested. Equal concentrations of fluorescently labeled WT (Oregon green) and M337V (Cy3) proteins were mixed in the presence of A(GU)₁₈ RNA. This led to the formation of both larger and small chain-like droplets that consisted of WT and M337V proteins (see e.g., FIG. 22). These results suggest that WT and mutant TDP-43 may assemble together to form condensates, however, WT protein may not transfer liquid-like properties to M337V complexes. Instead, mutant TDP-43 was able to disrupt WT LLPS behavior. These observations may be relevant to the ALS cases affected by the M337V mutation, caused by the heterozygous substitution c. 1009A>G, which has been independently found in families in a number of countries. The results demonstrate that one intact copy of TDP-43 is likely insufficient to prevent the maturation of liquid RNP granules to more solid structures in these patients. The assays established for these studies shed light on mechanisms essential for TDP-43 function that may be disrupted by ALS mutations and may underpin TDP-43-linked disease. The analysis also indicates that these mutations affect protein/gene function differently. The defects observed in the case of A321G and M337V may be caused by structural alterations that inhibit LLPS and/or result in condensates with gel or more solid-like properties. For this reason, mutant condensates may be more likely to increase fibrilization, as observed previously. These conclusions are also supported by previous reports of similar condensate clusters formed by the CTD harboring M337V. Interestingly, A321G and M337V are located within the alpha-helical region in the CTD. The observations that long GU-rich RNA is unable to promote liquid TDP-43 condensation in the case of these mutants aligns with earlier reports, highlighting the importance of alpha-helix-mediated intermolecular contacts on LLPS. This model warrants further investigation as it may not fully explain the observations with the Q331K mutation. Q331K also disrupts the CTD interactions of the isolated fragment, but behaves similar to WT in the assays with RNA-driven LLPS of full length TDP-43 (see e.g., FIG. 22), which is consistent with recently published work showing minimal differences in the cellular condensation properties of this mutant relative to WT.

### DISCUSSION

The self-assembly of TDP-43 plays a central role in determining protein function and cellular dynamics while at the same time, aberrant self-association may lead to protein aggregation and pathology associated with numerous neurodegenerative disorders. Herein was investigated the control of TDP-43 condensation by RNA molecules for their role as a principal TDP-43 interacting partner. Previous studies showed that full-length TDP-43 LLPS is inhibited in the presence of total yeast RNA, suggesting that RNA may prevent condensation. For the studies herein, a simplified system with full-length TDP-43 was used, and it was found that RNA strongly promotes TDP-43 phase separation in a sequence and length dependent manner. This process is mediated by specific binding, suggesting that TDP-43 condensation may be regulated by the number of proximal binding sites on RNA targets. However, at very high concentrations of specific-binding RNA this observation is reversed and this is in line with previous observations studying the CTD fragment condensation. Lower concentrations of GU-rich RNA may allow multiple TDP-43 molecule binding to each oligonucleotide, promoting droplet formation, whereas saturating RNA concentrations result in each RNA molecule binding to a single TDP-43 molecule. Similar to A(GU)₆ being unable to promote LLPS at 250 mM of NaCl, this will drive the system back toward a single-phase regime. The experiments in cell lysates showing that RNA composed of multiple TDP-43-binding sites promotes TDP-43 phase separation, whereas RNA composed of a single binding module does not induce TDP-43 granule formation, strongly supports the physiological relevance of this RNA-mediated function. Importantly, it was shown herein that specific interactions of the RNA-binding domains with GU-rich RNA are crucial to maintain liquid properties of the TDP-43 condensates. Furthermore, the results indicate that specific TDP-43 disease-linked mutations greatly impair this function of RNA, which may help explain the connection between disease-associated mechanisms and TDP-43 homeostasis.

The studies herein focused on RNA composed of sequences representative of TDP-43 targets found in cells, which are characterized by long GU-rich stretches. These RNA sequences that are predicted to bind multiple TDP-43 molecules are potent modulators of TDP-43 condensation. Increasing the number of TDP-43-binding modules enhances protein condensation, as seen by the size of droplets and increased enrichment of protein in the dense phase (see e.g., FIG. 14 and FIG. 17A-FIG. 17B). This process is impaired upon disruption of RNA binding using either RRM mutations (see e.g., FIG. 1A-FIG. 1B and FIG. 3), or when using RNA molecules with no measurable TDP-43-binding affinity (see e.g., FIG. 1A-FIG. 1B, FIG. 3, FIG. 5A-FIG. 5B, and FIG. 9A-FIG. 9C). The findings indicate that: (i) the ability of RNA to regulate TDP-43 phase separation depends on sequence-specific interactions between the RNA-binding domains of the protein and RNA; and (ii) the size of the TDP-43-binding region is a strong determinant of TDP-43 LLPS. These results suggest that TDP-43 LLPS may be predicted based on the sequence composition of RNA targets. Furthermore, this may provide insight into how TDP-43 function is modulated depending on whether or not phase separation is involved. These conclusions are supported by previous reports, where it was found that specific RNA sequences in the transcriptome and TDP-43 condensation are tightly linked, affecting RNA processing function of the transcripts involved. Among these RNA regions are long GU-rich sequences and the 3′ UTR of Tardbp, which contains CLIP34. In addition, previous reports suggested that long GU-repeat RNA binds multiple TDP-43 molecules cooperatively and that these interactions are important to maintain TDP-43 solubility. To further investigate whether the RNA-driven condensation is mediated by multivalent interactions, which characterize LLPS, domains that mediate TDP-43 oligomerization and self-assembly were disrupted.

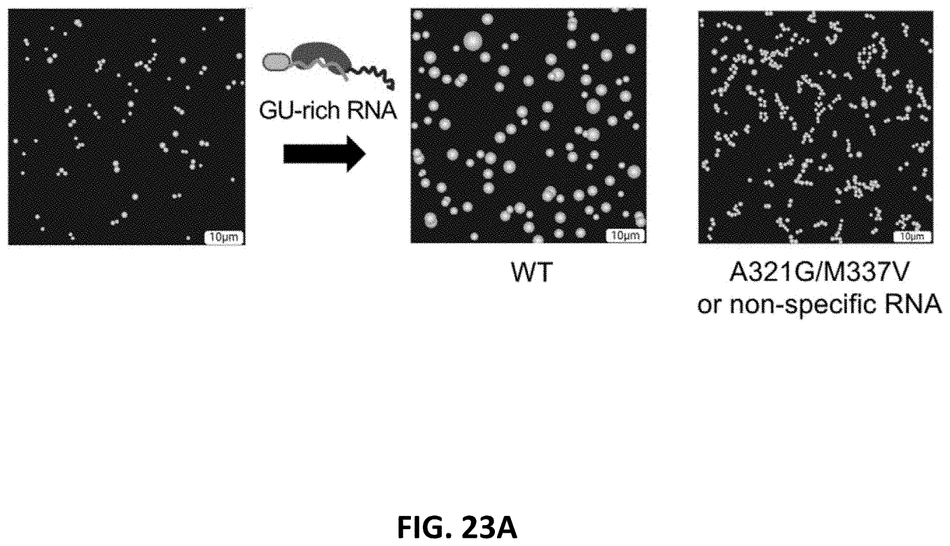

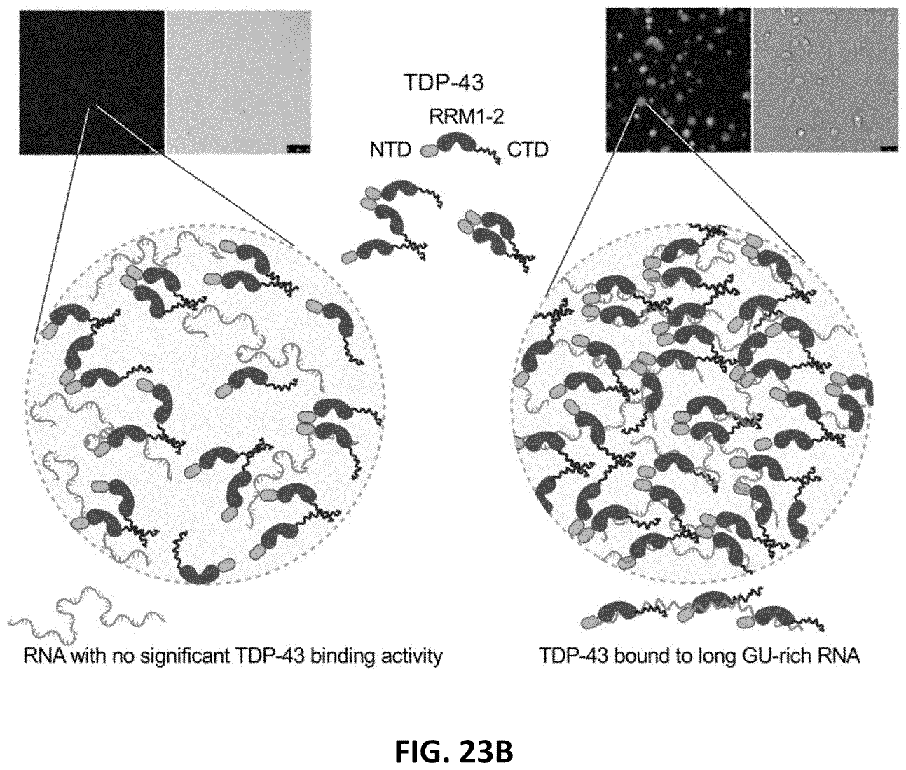

Mutations either in the amino terminal or carboxyl low complexity domain block droplet formation in the presence of RNA, under the conditions tested (see e.g., FIG. 18A-FIG. 18B). While no LLPS of these mutants was observed in these conditions, it is important to note that at different protein, salt, or RNA concentrations, it is certainly possible that these constructs would enter into a two-phase regime. In addition, the ALS-associated mutations A321G and M337V, which affect a conserved region in the CTD, show reduced liquid properties of the RNA-mediated complexes (see e.g., FIG. 20, FIG. 22, and FIG. 23A). Like the mutant fragments, the ALS mutations do not disrupt GU-rich RNA-binding affinity (see e.g., TABLE 2), but impair self-assembly and accelerate aggregation. These results underscore the importance of the interactions mediated by this C-terminal region during RNA-triggered condensation. A model in FIG. 23B is proposed whereby GU-rich RNA or RNA sequences that bind multiple TDP-43 molecules sact as a scaffold bringing multiple TDP-43 molecules in close proximity. This spatial organization may increase formation of the meshwork through N-terminal and CTD-mediated interactions highlighted in the model (see e.g., FIG. 23B). These interactions constitute a multivalent network by increasing the number of crosslinks between RNA-bound TDP-43. This is in contrast with conditions in which RNA molecules do not significantly interact with TDP-43 and fail to concentrate the protein. In this scenario, NTD and CTD interactions may still occur, however, the number of possible linked interactions is considerably less.

It has been shown that RNA exerts chaperone-like functions which increase solubility of RNA-binding proteins in cells, including in the case of TDP-43. Herein it was shown that RNA maintains the liquid properties within the droplets, decreasing maturation into solid-like assemblies (see e.g., FIG. 1B). This RNA activity is dependent on specific RNA binding and RNA composed of a single TDP-43-binding site, A(GU)₆, is sufficient to increase the liquid properties of TDP-43 droplets (see e.g., FIG. 1B). This observation is in agreement with previous findings that A(GU)₆ is a potent inhibitor of TDP-43 aggregation using the purified protein. Therefore, the RNA-bound state may stabilize the RRMs of TDP-43 in soluble conformations preventing the initial steps of protein aggregation that were previously shown to be triggered by RRM misfolding. These findings may provide insight into TDP-43 proteostasis in cells, particularly in the cytoplasm where TDP-43 pathology is predominantly found. The pathological inclusions in the cytoplasm may result from decreased RNA levels in this compartment relative to the nucleus, as previously suggested. Thus, RNA binding and recruitment to RNP granules may play a key role in maintaining soluble TDP-43 particularly in the cytoplasm. Cytosolic stress granules, which have been viewed as crucibles for TDP-43 pathology may actually be protective in preventing TDP-43 aggregation as they accumulate high levels of RNA molecules. This is supported by findings showing that TDP-43 aggregation is increased when the protein is excluded from stress granules. Based on the study herein, the dynamic properties and reversibility of stress granules and other ribonucleoprotein granules, such as axonal RNA transport granules may depend on specific TDP-43-RNA interactions.

In summary, the findings herein provide evidence for a model in which RNA binding drives TDP-43 phase separation and that this activity is modulated by the number of proximal protein-binding sites (see e.g., FIG. 23B). These findings carry important implications on the mechanisms by which TDP-43 controls RNA metabolism. Furthermore, this work lays the foundation for the development of new therapeutic strategies that utilize RNA molecules to alter TDP-43 phase properties and regulate protein homeostasis.

Materials and Methods

Reagents were purchased from Sigma unless otherwise noted. All experiments, unless otherwise noted were performed at room temperature (approximately 22° C.).

Expression and Purification of Recombinant TDP-43

Recombinant His6-SUMO N-terminally tagged TDP-43 was expressed in BL21(DE3) Escherichia coli cells from the SUMO-TDP43 plasmid (pET28b(+)-SUMO-TDP43). Mutants were generated by QuickChange site-directed mutagenesis (Agilent) using DNA oligonucleotides listed in TABLE 3.

TABLE 3
DNA oligonucleotide sequences used for mutagenesis.

| Name | DNA oligonucleotide sequence (5′→3′) |
|---|---|
| Y4R/E17R | See e.g., French RL, et al.* |
| ΔN | See e.g., French RL, et al.* |
| (102-414) | |
| ΔC | See e.g., French RL, et al.* |
| (1-269) | |
| RRM1-2 | See e.g., French RL, et al.* |
| (102-269) | |
| F147/149L | FW: GACTGGTCATTCAAAGGGGCTTGGCCTTGTTCGTTTTAC |
| | GGAATATG (SEQ ID NO: 14) |
| | RV: |
| | CATATTCCGTAAAACGAACAAGGCCAAGCCCCTTTGAATG |
| | ACCAGTC (SEQ ID NO: 15) |
| F229/231L | FW: CCAAGCCATTCAGGGCCCTTGCCCTTGTTACATTTG |
| | CAGATGATC (SEQ ID NO: 16) |
| | RV: GATCATCTGCAAATGTAACAAGGGCAAGGGCCCTGAA |
| | TGGCTTGG (SEQ ID NO: 17) |
| K181E | FW: GTGTGACTGCAAACTTCCTAATTCTGAGCAAAGCCAAGA |
| | (SEQ ID NO: 18) |
| | RV: TCTTGGCTTTGCTCAGAATTAGGAAGTTTGCAGTCACAC |
| | (SEQ ID NO: 19) |
| A315E | FW: GATGAACTTTGGTGAGTTCAGCATTAATC (SEQ ID NO: |
| | 20) |
| | RV: GATTAATGCTGAACTCACCAAAGTTCATC (SEQ ID NO: |
| | 21) |
| A315T | French RL, et al.* |
| A321G | FW: TTTGGTGCTTCAGCATTAATCCAGGCATGATGGCTGCCG |
| | CCCAGGCAG (SEQ ID NO: 22) |
| | RV: CTGCCTGGGCGGCAGCCATCATGCCTGGATTAATGCTG |
| | AACGCACCAAA (SEQ ID NO: 23) |
| Q331K | FW: GCCGCCCAGGCAGCACTAAAGAGCAGTTGGGGTATGA |
| | TG (SEQ ID NO: 24) |
| | RV: CATCATACCCCAACTGCTCTTTAGTGCTGCCTGGGCGGC |
| | (SEQ ID NO: 25) |
| M337V | French RL, et al.* |
| A382T | FW: CTCTAATTCTGGTGCAACAATTGGTTGGGGATCAG (SEQ |
| | ID NO: 26) |
| | RV: CTGATCCCCAACCAATTGTTGCACCAGAATTAGAG (SEQ |
| | ID NO: 27) |

*French RL, et al. Detection of TAR DNA-binding protein 43 (TDP-43) oligomers as initial intermediate species during aggregate formation. J Biol Chem 294, 6696-6709 (2019).

The protocol for protein preparation was modified from previously described methods. Briefly, induction was carried out with 0.3 mM of isopropyl β-D-thiogalactopyranoside (IPTG) and cells were grown at 16° C. for 16 h. Cell pellets were resuspended in lysis buffer (1 M of NaCl, 50 mM of HEPES, pH 7.5, 2% TRITON X-100, 5% glycerol, SIGMAFAST EDTA-free protease inhibitor cocktail, 2.5 μg/ml of lysozyme, 10 μg/ml of DNase I, 1 mM (tris(2-carboxyethyl) phosphine) (TCEP)). Lysates were centrifuged for 20 min at 20,000 g at 4° C. following sonication on ice. Protein was bound to nickel-nitrilotriacetic acid resin (McLab), and the resin was loaded into a column for purification (AKTA-Start, GE Healthcare). The column was washed with wash buffer 1 (500 mM of NaCl, 50 mM of HEPES, pH 7.5, 5% glycerol, 1 mM of TCEP), followed by wash buffer 2 (500 mM of NaCl, 50 mM of HEPES, pH 7.5, 5% glycerol, 50 mM of Imidazole, 1 mM of TCEP), and eluted in elution buffer (500 mM of NaCl, 50 mM of HEPES, pH 7.5, 5% glycerol, 300 mM of Imidazole, 5 mM of DTT). Protein was flash frozen in liquid nitrogen and stored in aliquots at −80° C.

Purification of Untagged TDP-43

This protocol was modified from previously described methods. As above, His6-SUMO-TDP-43 was expressed in BL21(DE3) Escherichia coli cells. Cells were lysed in lysis buffer. Lysates were sonicated and centrifuged. Protein was bound to nickel-nitrilotriacetic acid resin, washed with wash buffer 1, wash buffer 2, and again with wash buffer 1. Recombinant His6-ULP1 was added to the protein-bound resin and incubated at room temperature for 30 min. The supernatant containing TDP-43 was collected.

Oligonucleotide Development

All oligonucleotides were modified with a 2′OMe group as well as a phosphorothioate backbone unless otherwise noted. Oligonucleotides were synthesized and modified by Integrated DNA Technologies (IDT).

LLPS Assay

Thawed recombinant protein was centrifuged for 35 min at 98,400 g at 4° C. to remove any preformed aggregates and was diluted to the required starting concentration using elution buffer. LLPS buffer (50 mM of HEPES, pH 7.5, 5% glycerol, 5 mM of DTT) containing appropriate NaCl and RNA concentrations was spotted onto a Lab-Tek Chambered #1.0 borosilicate cover glass slide, followed by the addition of protein. Droplets were imaged by brightfield and fluorescence microscopy using a Leica TCS SP5 confocal microscope with an HCX APO U—V-I 100.0×/1.30 oil immersion objective and 2.5×digital zoom at room temperature. For experiments involving fluorescently labeled protein, recombinant protein was labeled with Oregon Green 488 or Cy3 maleimide dye (Thermo Fisher Scientific, OG-TDP-43, Cy3-TDP-43) according to manufacturer's protocol, purified with Zeba Spin columns (Thermo Fisher Scientific), and mixed with unlabeled protein in a ratio of 1:10. For experiments involving fluorescently labeled RNA, Alexa Fluor 594-labeled RNA was mixed with unlabeled RNA in a ratio of 1:10. Images were taken approximately 45 min after beginning the reaction, unless otherwise noted. OG-TDP-43 was excited using a 488 nm laser line, Cy3-TDP-43 was excited using a 514 nm laser line, and labeled RNA was excited using a 543 nm laser line. For droplet area measurements, droplet regions of interest were manually selected and analyzed using ImageJ. Fluorescent channel intensity values were universally increased within experiments by the same amount for each image, and brightfield images were linearly contrasted using Leica LAS AF software.

Fluorescence Recovery after Photobleaching

TDP-43 and RNA liquid droplets were first generated as described above. Ten baseline images were taken in 0.376 s intervals, and ROIs were bleached for 3.76 s using 30% laser power from a 488 nm laser line. Eleven post-bleach images were then taken in 0.376 s intervals, followed by 4 images taken in 20 s intervals. Data points were normalized using the equation Fr₍ₜ₎=(I₍ₜ₎)−I_{bleach})/(I_{pre-bleach}−I_{bleach}) where Fr₍ₜ₎ is fluorescence recovery at a given time point, I₍ₜ₎ is intensity at a given time point, I_{bleach} is the intensity immediately following bleaching, and I_{pre-bleach} is the prebleach intensity.

High Salt Droplet Reversal Assay

TDP-43/RNA droplets were formed as described above. As soon as droplets began to settle on the glass, high salt buffer (4 M of NaCl, 50 mM of HEPES, pH 7.5, 5% glycerol, 5 mM of DTT) was added to a final NaCl concentration of 1.3 M in order to promote dissolution of condensates.

Turbidity Assay

Thawed recombinant protein was centrifuged for 35 min at 98,400 g at 4° C. and was diluted to the required starting concentration using elution buffer. LLPS buffer containing appropriate RNA concentration was added to clear 96-well clear-bottom plates (Greiner Bio-One), followed by the addition of protein. Plates were read at room temperature for 1 h using a SpectraMax i3 microplate reader (Molecular Devices). Turbidity was measured as maximum absorbance at 600 nm λ.

Measurement of Light Phase TDP-43 Concentration

Thawed recombinant protein was centrifuged for 35 min at 98,400 g at 4° C. and was diluted to the required starting concentration using elution buffer. LLPS buffer containing appropriate RNA concentration was mixed with protein and incubated for 30 min. The samples were centrifuged at 21,000 g for 10 min to pellet the dense phase. The concentration of protein in the supernatant, or light phase (Cₒᵤₜ), was then measured by Bradford assay using a Nanodrop spectrophotometer.

Fluorescence Anisotropy Assay

Recombinant TDP-43 wild-type (WT) and constructs previously purified were serially diluted in a 1:2 ratio (from 75 to 0 nM) into a 300 mM of NaCl, 10 mM of Tris (pH 8.0), 5% glycerol, 5% sucrose, 1 mM of TCEP buffer solution. A(GU)₆ RNA labeled with fluorescein isothiocyanate (FITC) from IDT was used at a final concentration of 0.6, 1.3, 2.5, and 5 nM. The protein preparations were mixed with A(GU)₆-FITC and added in triplicate in a 384-well black flat bottom plate (Corning) protected from light. The anisotropy measurements were performed in a Biotek SYNERGY Neo2 (BioSPX) plate reader with excitation and emission wavelengths of 480 and 520 nm, respectively. To assess the binding curves, the data were fitted as previously described. Data analysis was performed using GraphPad Prism 5.

Electromobility Shift Assay

The EMSA protocol was adapted from previously described methods. Briefly, purified full-length TDP-43 was centrifuged 98,400 g, 30 min, 4° C. Varying protein (0-7 μM range) was incubated with 10 nM of CLIP-34 oligonucleotide labeled with 5,700 nm IR, 5′ minutes on ice and 25′ at room temperature in binding buffer (150 mM of NaCl; 10 mM of Tris pH 8; 2 mM of MgCl2; 5% glycerol; 1 mM of DTT). Electrophoresis of 6% polyacrylamide gels was performed at 100 V. Images were acquired using the LI-COR Odyssey platform.

Cell Constructs

HA-mEGFP-TDP constructs were created by subcloning TDP-43 cDNA into the mEGFP-C1 vector (Addgene) using the restriction enzymes XhoI and HindIII. The resulting mEGFP-TDP-43 gene was then subcloned into the pCDNA5 (Thermo) vector using the restriction enzymes EcoRV and NotI downstream of the HA-tag.

Cell Culture and Induction of mEGFP-Tagged TDP-43

HEK Flp-In™ T-REX™ 293 cells (Thermo) were grown in Dulbecco's modified Eagle's medium (DMEM) supplemented with 10% fetal bovine serum. Stable incorporation of mEGFP-TDP-43 was achieved using the pcDNA™5/FRT/TO Vector system (Thermo). Cells were plated in cell-culture treated 10-cm dishes. Expression of GFP-tagged TDP-43 protein was induced at 70% confluency by addition of 1 μg/ml of tetracycline for 24 h prior to harvesting.

Cell Collection, Lysis, and Induction of LLPS Using Cell Lysates

The following protocol was adapted from previously described methods. Cells were washed with PBS and pelleted by centrifugation at 300 g for 5 min. PBS was aspirated and cell pellets were frozen at −80° C. Prior to use, pellets were thawed at room temperature for 2 min. 200 μl of lysis buffer (50 mM of HEPES, pH 7.5, 500 mM of NaCl, 0.5% NP-40, 5% glycerol, 5 mM of DTT, and SigmaFast EDTA-free protease inhibitor cocktail) was added to cell pellets. Pellets were lysed by pipetting up and down and transferred to Eppendorf tubes, incubated for 3 min at room temperature, centrifuged at 21,000 g for 5 min at room temperature and the supernatant was collected. Microscopy and turbidity experiments were conducted as described above, with cell lysate as 10% by volume added as the final component.

### Example 2: RNA-Based Approach to Decrease Tdp-43 Pathology

The following example describes the use of specific RNA oligonucleotides to prevent TDP-43 aggregation and decrease neurotoxicity.

There are ongoing efforts to treat and cure neurodegenerative disorders linked to TAR DNA-binding protein (TDP-43) pathology, or TDP-43 proteinopathies. The accumulation of TDP-43 aggregates and associated loss of function are key features of Amyotrophic Lateral Sclerosis (ALS), also known as Lou Gehrig's disease, frontotemporal dementia (FTD or FTLD-TDP-43) and limbic-predominant age-related TDP-43 encephalopathy (LATE). In addition, TDP-43 inclusions are present in approximately 50% of Alzheimer's disease cases and other neurodegenerative disorders including multisystem proteinopathy and chronic traumatic encephalopathy. These are incurable, late onset disorders that afflict a rapidly growing segment of the global population. Described herein is the development of new therapeutic molecules to reduce neurodegeneration and toxicity in these TDP-43-associated disorders.

TDP-43 inclusions accumulate in neurons and glial cells in the brain and spinal cord of affected individuals. These deposits coincide with a dramatic reduction of normal nuclear TDP-43 detection, implying a loss of function upon aggregation. This is viewed as a major factor impeding neuronal function as TDP-43 is known to be essential for development and survival. Whether disease results from aggregate toxicity, from sequestration of functional TDP-43 into aggregates, or from a combination of both mechanisms is yet unclear. Either way, reducing the accumulation of TDP-43 aggregates is a major goal to decrease neurodegeneration. TDP-43 binding to RNA strongly inhibits its aggregation depending on nucleotide sequence composition (see e.g., Example 1). These findings strongly suggest that RNA acts as a chaperone maintaining TDP-43 solubility. Thus, specific RNA molecules may be used to prevent TDP-43 aggregation and decrease neurotoxicity. This model has been tested using established methods using purified TDP-43. As described herein, new molecules will be screened and identified to effectively reduce TDP-43 aggregation and its associated pathology.

Specific RNA molecules may be used to decrease TDP-43 aggregation, which will reduce toxicity and restore protein function. Specific RNA molecules have been designed and tested for their ability to prevent TDP-43 aggregation without causing loss of protein function or cell toxicity. These experiments have been carried out using various models of TDP-43 pathology. The study is divided as follows:

1. Establish RNA-based methods to block TDP-43 aggregation. Identify specific RNA molecules that efficiently inhibit TDP-43 aggregation, increase solubility and restore protein function. For these experiments, purified TDP-43 was used cultured human cell-based models with established assays.

2. Determine whether RNA inhibits intracellular TDP-43 aggregate seeding and propagation. The ability of RNA to inhibit intracellular TDP-43 aggregate seeding by pre-formed fibrils was tested. This work was performed using an established reporter cell line and seeding protocols to quantify aggregate spread. inhibits its aggregation depending on nucleotide sequence composition. These findings strongly suggest that RNA acts as a chaperone maintaining TDP-43 solubility and supports that specific RNA molecules may be used to prevent TDP-43 aggregation and decrease neurotoxicity. This model has been tested using established methods using purified TDP-43.

1. Establish RNA-Based Methods to Block TDP-43 Aggregation.

1a: Identification of RNA Oligonucleotides that Inhibit Aggregation of Purified TDP-43.

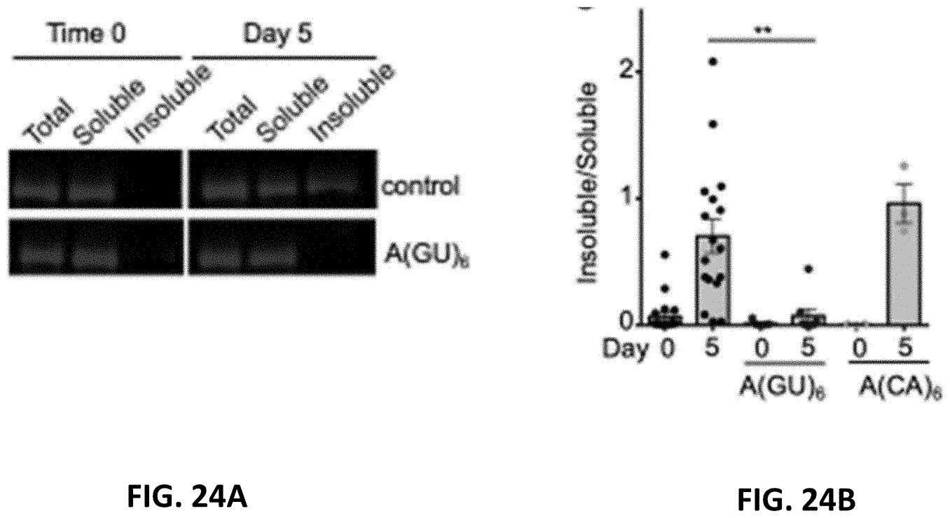

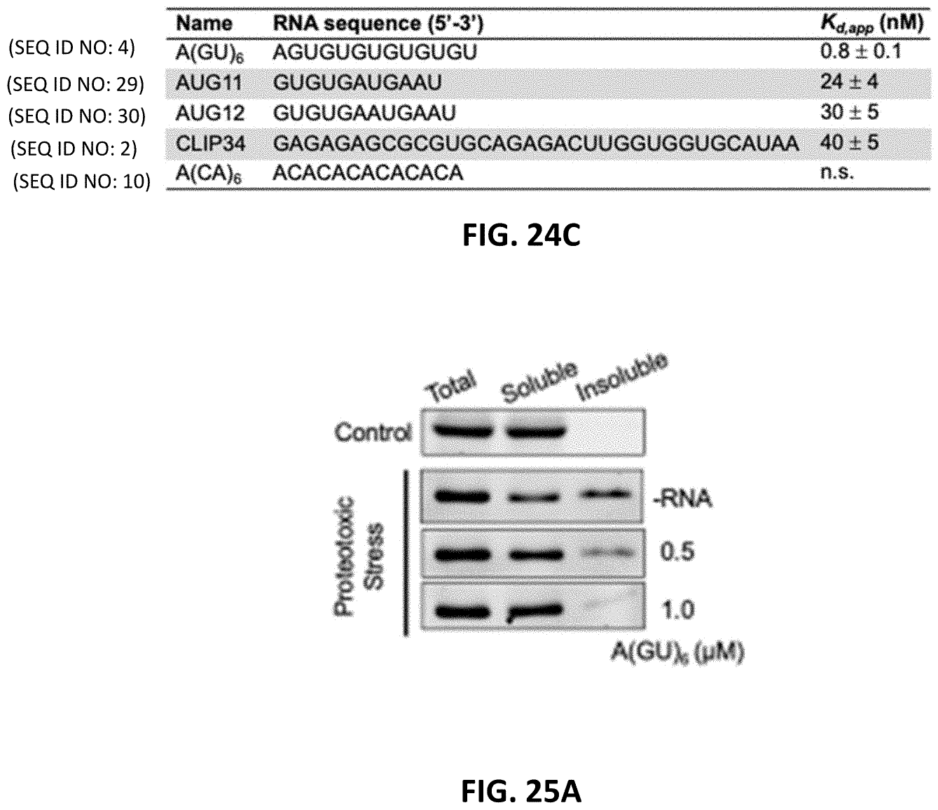

Earlier studies suggested that the ability of RNA molecules to inhibit TDP-43 aggregation depends on specific binding. Their ability to decrease TDP-43 aggregation was compared using a purified protein system and cell-based models. The results show that guanine and uridine-rich RNA oligonucleotides are most efficient at preventing TDP-43 aggregation. The aggregation of purified TDP-43 was examined comprising separating soluble protein from aggregate complexes (see e.g., FIG. 24A-FIG. 24C). Semi-denaturing detergent agarose gel electrophoresis (SDD-AGE) was previously optimized to study TDP-43 aggregate intermediates. Using this technique, a dramatic decrease of TDP-43 aggregation was previously observed upon addition of (GU)₆ (see e.g., French et al. (2019)). These results were supported by the quantitative comparison of soluble to insoluble TDP-43 fractions taken at time 0 and five days after triggering aggregation (see e.g., FIG. 24A-FIG. 24B). Here, addition of A(GU)₆ RNA maintained TDP-43 in the soluble fraction, inhibiting accumulation of the misfolded species. As control, an RNA oligonucleotide of similar length was used, A(CA)₆, which previously showed no significant binding activity for TDP-43. Addition of A(CA)₆ did not significantly alter the ratio of insoluble/soluble TDP-43. These initial observations strongly suggest that the ability of RNA to maintain TDP-43 solubility directly correlates with specific binding. RNA molecules with the highest binding affinity may be the strongest inhibitors of aggregation. A group of 20 RNA oligonucleotides with different binding affinity for TDP-43 was screened, as measured by fluorescence anisotropy (see e.g., FIG. 4 and FIG. 24C). GU-rich RNA oligonucleotides with affinities in the nanomolar range were then tested using these assays and similar effect on decreasing TDP-43 aggregation was observed as with A(GU)₆ RNA.

Next, whether RNA binding could decrease the conversion of TDP-43 biocondensate fibrilization or maturation into irreversible aggregates was tested. These studies were based on the paradigm that intracellular organization of RNA binding proteins into ribonucleoprotein (RNP) granules is mediated by a process of condensation or phase separation. Based on evidence emerging over the last decade, these protein and RNA-rich granules are formed via liquid-liquid phase separation (LLPS) that often show dynamic, liquid-like properties. The high concentration of the components while retaining dynamic properties within the droplets may be important to determine function, cellular organization and rapid response to cellular stimuli. Numerous studies also suggest that defects in LLPS homeostasis are associated with disease, such as in the case of TDP-43, FUS (Fused in sarcoma) and other RNA binding proteins linked to neurodegeneration. Specifically, conversion of the condensates into fibrils or complexes with solid-like properties may lead to the accumulation of protein aggregates associated with pathology. Previous studies strongly suggested that biocondensate maturation is the first step in TDP-43 aggregation (see e.g, French et al. (2019)). TDP-43 droplets fail to coalesce for up to 2 hours of incubation and, instead, form clusters or chain-like assemblies, suggesting that these complexes are more viscous and have gel or solid-like properties (see e.g., FIG. 1A). Whether association with A(GU)₆ RNA oligonucleotide could alter the dynamic properties of TDP-43 droplets was then investigated. Consistently, addition of this RNA oligonucleotide dramatically increased the size of TDP-43 droplets compared to no RNA conditions through dynamic fusion (see e.g., FIG. 1B). The TDP-43 droplets in the presence of A(GU)₆ maintained liquid-like properties, as shown by fluorescence recovery after photobleaching (FRAP) (not shown). In contrast, the non-specific A(CA)₆ RNA or RNA binding-deficient TDP-43 mutants showed no significant changes compared to no RNA control. These results suggest that RNA binding increases the dynamic and liquid properties of TDP-43, depending on RNA sequence and specific interactions. RNA binding may prevent the conversion of TDP-43 droplets into solid-like complexes. This work also suggests that RNA oligonucleotides may effectively block TDP-43 pathology as early as during the initial misfolding stage.

1b: Test RNA-Driven Inhibition of Cellular TDP-43 Aggregation.

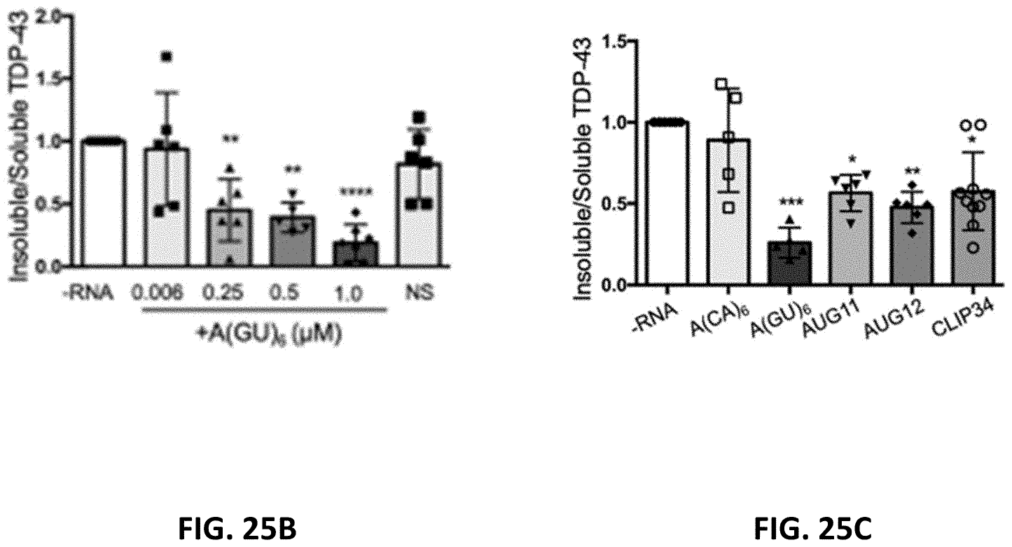

Next, it was investigated whether specific RNA binding may also promote TDP-43 solubility in human cells. For this established assays were used to promote proteotoxic stress and trigger TDP-43 aggregation. Human embryonic kidney cells (HEK293) were treated with MG132 to inhibit the ubiquitin proteosomal system combined with short exposure to oxidative stress triggered by sodium arsenite. Extraction of soluble and insoluble fractions from the cell lysate showed the expected increase of TDP-43 in the insoluble fraction under proteotoxic conditions (see e.g., FIG. 25A). Transfection of A(GU)₆ RNA prior to stress treatment resulted in a significant decrease of insoluble TDP-43, compared to no RNA control or non-specific RNA (NS) (see e.g., FIG. 25B). The anti-aggregation activity of A(GU)₆ RNA on TDP-43 was dose-dependent. Transfection of other GU-rich RNA molecules that bind TDP-43 with apparent dissociation constants (K_{d}) in the nanomolar range (see e.g., FIG. 24C) also inhibited TDP-43 aggregation significantly compared to control (see e.g., FIG. 25C). Conversely, the non-specific A(CA)₆ RNA did not show a significant effect on TDP-43 aggregation relative to control. These results strongly indicate that RNA molecules that specifically bind TDP-43 are capable of acting as chaperones and prevent TDP-43 misfolding in human cells.

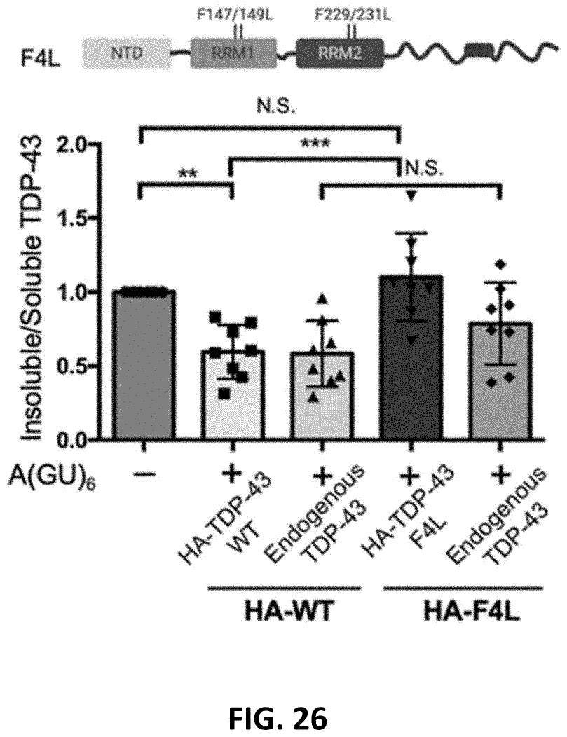

To further confirm that RNA stabilizes TDP-43 solubility through specific binding, cells expressing the RNA binding-deficient TDP-43 mutant, F147/149/229/231L (F4L), were studied. In this case, (GU)₆ treatment was not able to rescue F4L aggregation (see e.g., FIG. 26). Interestingly, greater F4L aggregation was measured in cells not treated with RNA, compared to wild-type (not shown). Similarly, endogenous TDP-43 showed greater aggregation in F4L expressing cells, relative to WT cells. These findings suggest that the inability to bind RNA strongly impairs TDP-43 solubility. Moreover, the presence of F4L noticeably disrupts the homeostasis of normal TDP-43 expressed in the same cells, perhaps through a prion-like mechanism. Collectively, this data provides strong evidence that the chaperone function of RNA is correlated with its binding affinity to TDP-43, as predicted from our studies using purified protein.

During the design of this study, the possibility that sequences that bind TDP-43 with tight binding affinity (low nanomolar range), as (GU)₆, may compete with TDP-43 RNA binding partners and inhibit RNA processing was considered. To test this, the effect of RNA treatment on TDP-43 autoregulatory function was measured in cells, using well-established assays. Data shows no significant cell toxicity under the conditions tested. Moreover, (GU)₆ treatment did not significantly alter TDP-43 autoregulation, suggesting that GU-rich RNA does not significantly affect physiological TDP-43 activity in cells. To further test whether RNA treatment unintendedly decreases TDP-43 function, changes in the processing of known TDP-43 targets will be analyzed. In addition, it was found that RNA treatment did not significantly change TDP-43 cellular localization. Both RNA and control treated cells showed diffuse nuclear TDP-43 combined with characteristic nuclear foci and no increase in cytoplasmic detection. Furthermore, initial studies were performed to test whether (GU)₆ could alter the function of RNA binding proteins other than TDP-43. The formation of ribonucleoprotein (RNP) granules and their localization in (GU)₆-treated cells was surveyed and compared to control treated cells. More specifically, Cajal bodies (coilin), nucleoli (fibrillarin), splicing speckles (SC35), and stress granules (TIA-1) were visualized. The results show no significant changes in RNP granule assembly or localization, suggesting that (GU)₆ RNA does not alter RNA binding protein behavior in general, under the conditions tested.

2. Determine Whether RNA Inhibits Intracellular TDP-43 Aggregate Seeding and Propagation.

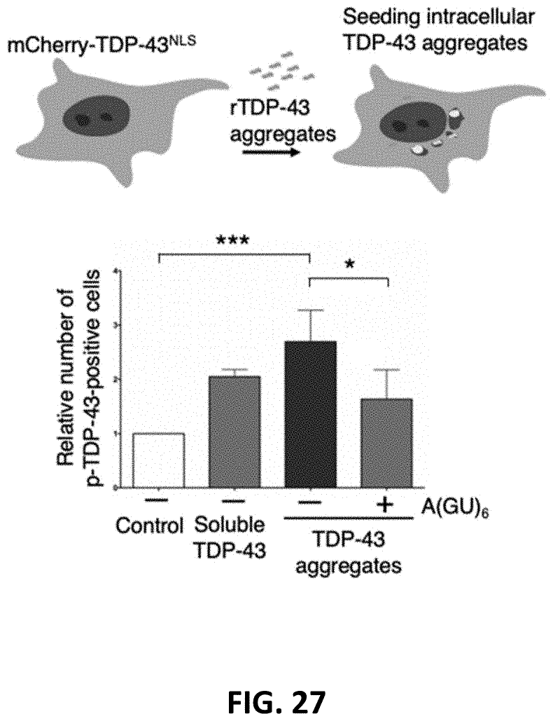

Recently, it was shown that transfection of human cells with recombinant TDP-43 aggregates results in a 3-fold increase in cellular TDP-43 cytoplasmic aggregation relative to treatment with soluble protein. Indeed, additional recent studies show that frontotemporal dementia (FTD)-derived, TDP-43-positive extracts spread TDP-43 pathology in mouse brain. The ability of TDP-43 aggregates to spread intracellularly in prion-like fashion is a strong indication that TDP-43 inclusions are toxic and support a gain of function mechanism in pathogenesis. Herein is examined the potential of RNA-based tools to prevent disease progression by targeting a central mechanism in the spread of TDP-43 pathology. Using an established cellular reporter, whether (GU)₆ RNA was able to prevent propagation of TDP-43 inclusions was tested, and it was found that intracellular seeding triggered by pre-formed TDP-43 aggregates significantly decreased in the presence of (GU)₆ RNA (see e.g., FIG. 27). Two additional models were recently developed to study cellular TDP-43 aggregate seeding and spread of pathology. One approach is to analyze TDP-43 aggregate propagation in a primary neurons derived from mouse brain cortex. The other technique uses recently obtained cell lines that are used to sense TDP-43 aggregation on the basis of Foster resonance energy transfer (FRET). These cell lines were used to detect TDP-43 aggregation and seeding using frontotemporal dementia extracts and pre-formed aggregates from purified TDP-43. Based on promising results with the FRET sensor line, these will be used to test the effect of RNA oligonucleotide treatment on TDP-43 aggregate seeding. The advantage of this method compared to the previously developed sensor cell line is that FRET analysis may be easily adapted for medium or high throughput screening.

### Example 3: Antisense Oligonucleotides (ASOs) to Decrease Tdp-43 Pathology

The following example describes antisense oligonucleotides (ASOs) and their modifications currently being tested for ability to bind TDP-43.

There is mounting evidence showing that the RNA binding domains of TDP-43 are highly unstable, play a major role in TDP-43 aggregation and that specific RNA sequences stabilize the soluble form of the protein.

There is also data showing differences in the binding affinity of TDP-43 for modified vs non-modified RNA in a sequence specific manner.

| | | ASO sequence with | | |
|---|---|---|---|---|
| | ASO sequence | modifications | | Target |
| Name | (5′→3′) | (5′→3′) | Modifications | position |
| A(GU)₆ | AGUGU | mA*mG*mU*mG*mU* | PS; 2-OMe; | RNA |
| 2OMe Bait | GUGUGUGU | mG*mU*mG*mU*mG* | fully modified; | binding |
| (SEQ ID | | mU*mG*mU | HPLC | domains |
| NO: 6) | | | purification; | |
| | | | Na⁺ Salt | |
| | | | exchange | |
| TDP43 bait | GAGAGAGCG | mG*mA*mG*mA*mG | PS; 2-OMe; | RNA |
| 2OMe (clip- | CGUGCAGAGA | *mA* | fully modified; | binding |
| 34) (SEQ | CUUGGUGGU | mG*mC*mG*mC*mG | HPLC | domains |
| ID NO: 7) | GCAUAA | *mU | purification; | |
| | | *mG*mC*mA*mG*mA* | Na⁺ Salt | |
| | | mG*mA*mC*mU*mU* | exchange | |
| | | mG*mG*mU*mG*mG* | | |
| | | mU*mG*mC*mA*mU* | | |
| | | mA*mA | | |
| Bait Scram | UAGGAGGGUC | mU*mA*mG*mG*mA* | PS; 2-OMe; | Control |
| 2OMe (clip- | AGAACUGAGA | mG*mG*mG*mU*mC* | fully modified; | |
| 34) (SEQ | GUGGUACAU | mA*mG*mA*mA*mC* | HPLC | |
| ID NO: 28) | CGCGG | mU*mG*mA*mG*mA* | purification; | |
| | | mG*mU*mG*mG*mU* | Na⁺ Salt | |
| | | mA*mC*mA*mU*mC* | exchange | |
| | | mG*mC*mG*mG | | |
| A(GT)₆ | AGTGTGTG | /52MOErA/*/i2MOErG/ | PS except | RNA |
| MOE (SEQ | TGTGT | i2MOErT/*/ | linkages 2 and | binding |
| ID NO: 8) | | i2MOErG/i2MOErT/*/ | 4 from ends; | domains |
| | | i2MOErG/*/ | 2-MOE; fully | |
| | | i2MOErT/*/2MOErG/*/ | modified; | |
| | | i2MOErT | HPLC | |
| | | /i2MOErG/*/i2MOErT/ | purification; | |
| | | i2MOErG/*/ | Na⁺ Salt | |
| | | 32MOErT/ | exchange | |
| A(GU) | AGUGUGU | /52MOErA/*/i2MOErG/ | PS except | RNA |
| mixed | GUGUGU | mU*/i2MOErG/mU*/ | linkages 2 and | binding |
| MOE OMe | | i2MOErG/*mU*/ | 4 from ends; | domains |
| (SEQ ID | | i2MOErG/*mU/ | 2-MOE and 2- | |
| NO: 9) | | i2MOErG/mU/i2MOErG/ | OMe mixed; | |
| | | mU | fully modified; | |
| | | | HPLC | |
| | | | purification; | |
| | | | Na⁺ Salt | |
| | | | exchange | |
| (CA)₆_Ome | ACACAC | mC*mA*mC* | PS; 2-OMe; | Control |
| (SEQ ID | ACACACA | mA*mC*mA* | fully modified; | |
| NO: 29) | | mC*mA*mC* | HPLC | |
| | | mA*mC*mA | purification; | |
| | | | Na⁺ Salt | |
| | | | exchange | |
| Scram | TCATTTGCTT | | 2′MOE fully | Control |
| (SEQ ID | CATACAGG | | modified | |
| NO: 30) | | | | |

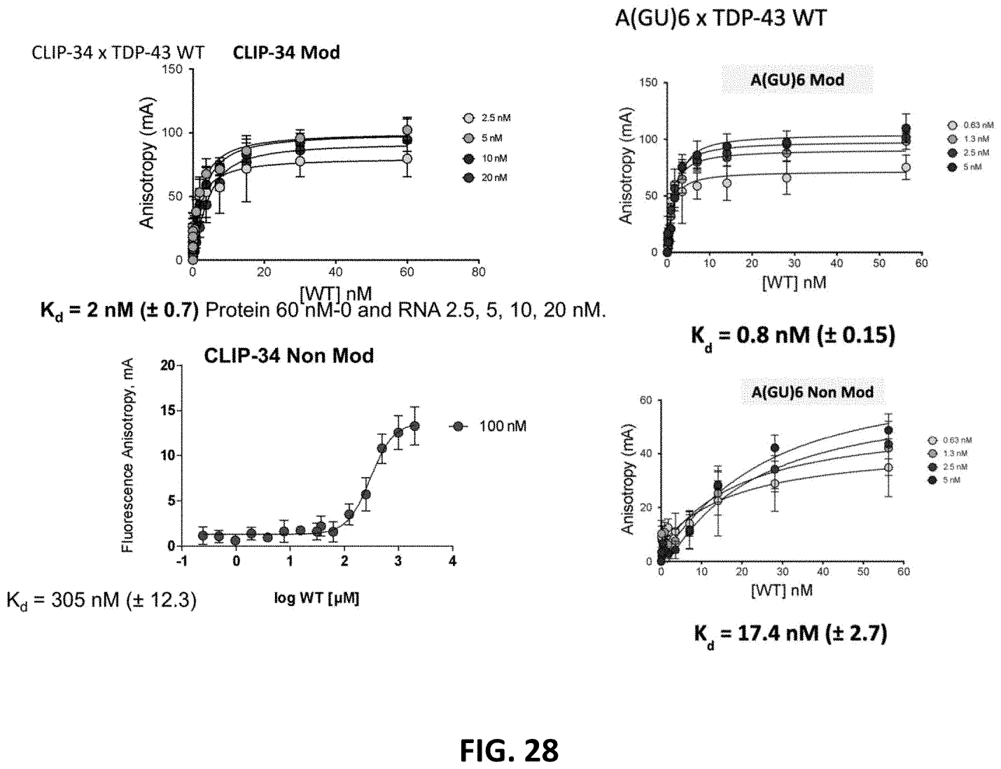

TDP-43 binding affinity to synthetic RNA was dramatically modulated by chemical RNA modifications (see e.g., FIG. 28). Fluorescence anisotropy was performed for modified CLIP-34 (e.g., antisense oligonucleotide), unmodified CLIP-34, modified A(GU)₆, and unmodified A(GU)₆ incubated with wild-type TDP-43. Modifications to CLIP-34 or A(GU)₆ resulted in a dramatically lower Kd value compared to their unmodified counterparts.

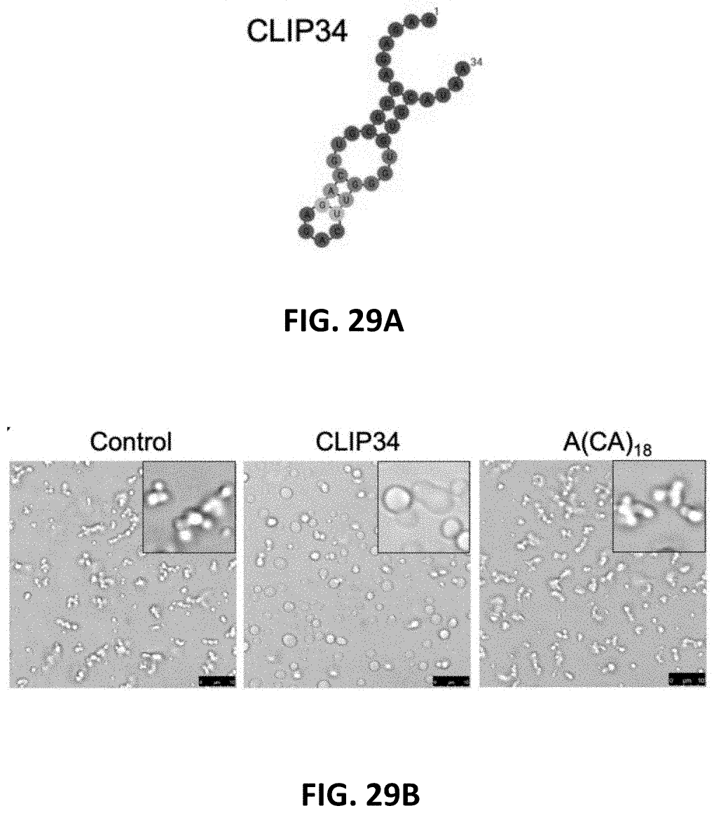

CLIP-34 was also shown to specifically increase the liquid properties of TDP-43 condensates and prevents formation of solid-like fibrils (see e.g., FIG. 29A-FIG. 29B).

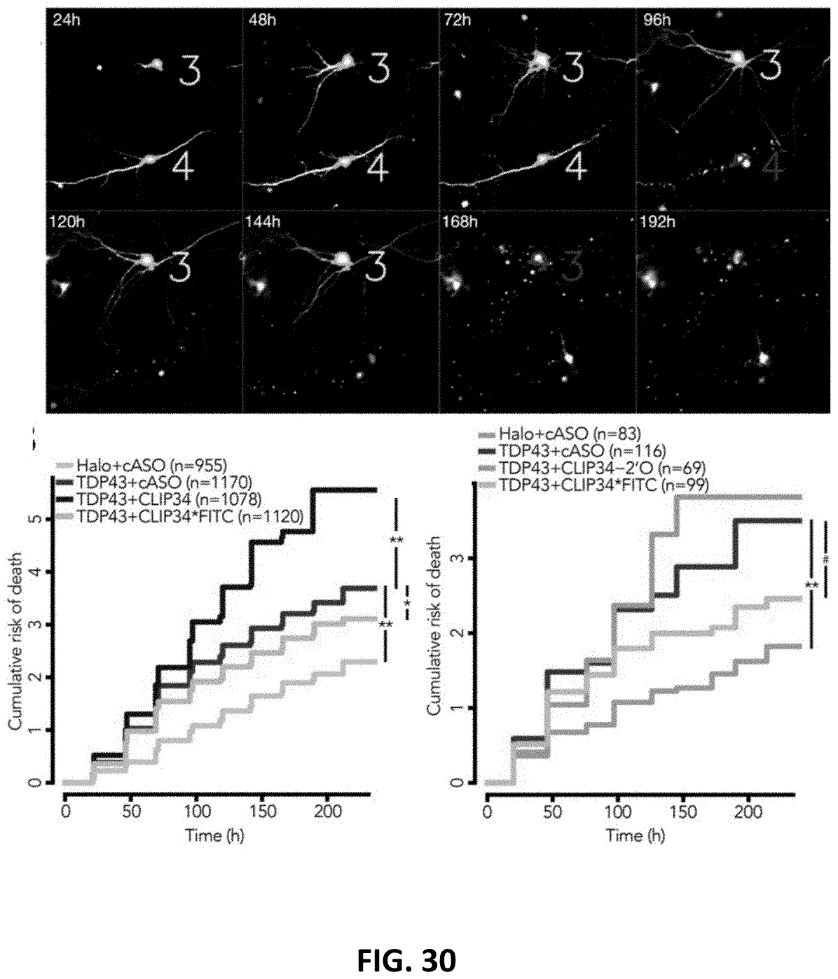

A specific effect of CLIP-34 was also demonstrated in increasing survival of a neuronal model of TDP-43-mediate toxicity. CLIP-34 ASOs displayed distinct effects on the survival of TDP43-expressing primary rodent neurons (see e.g., FIG. 30).

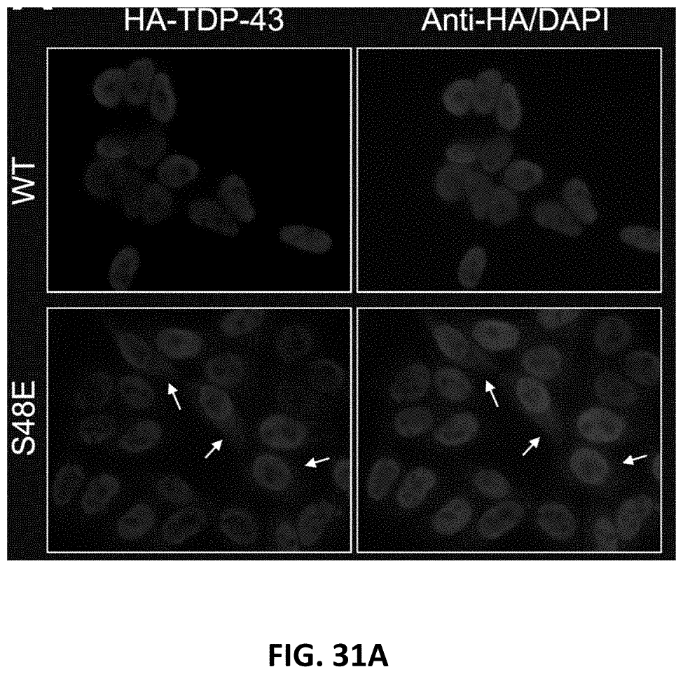

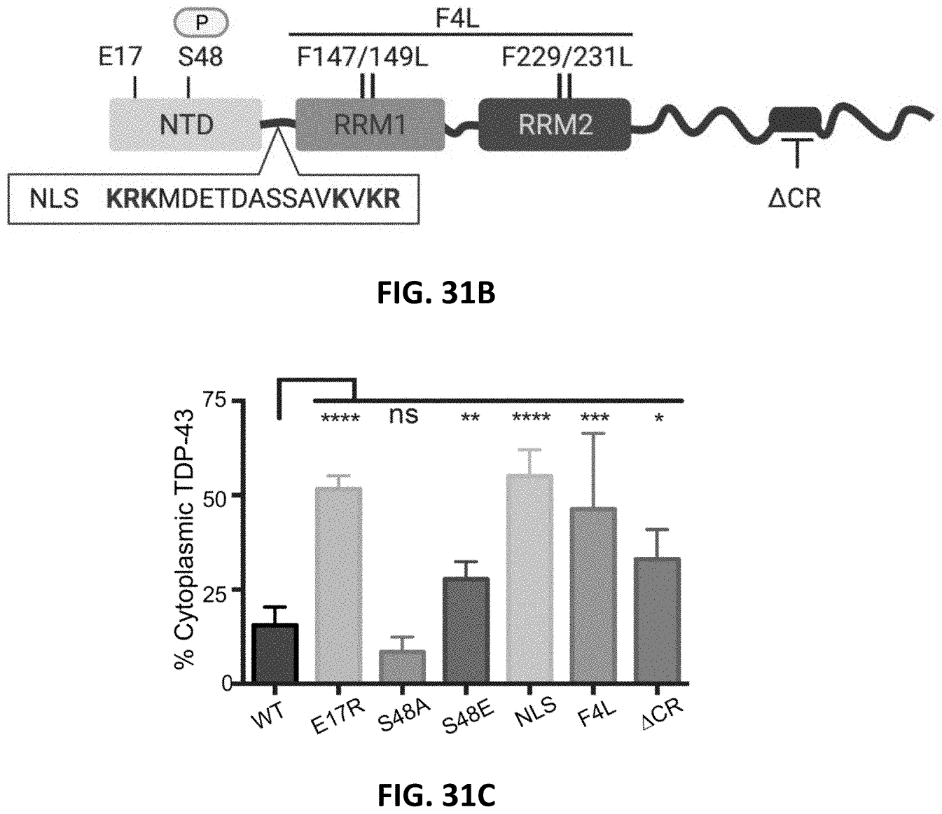

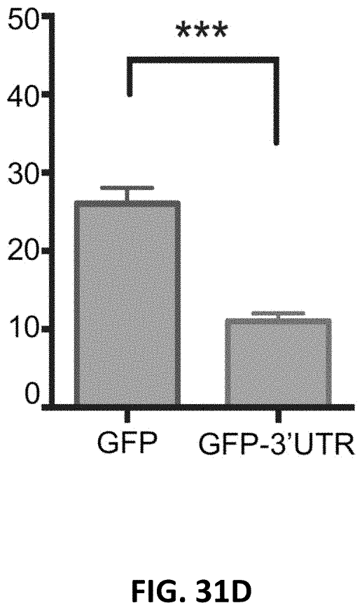

Specific RNA sequence binding to TDP-43 was also found to modulate the nuclear-cytoplasmic transport of the protein, which is tightly linked to pathology. WT TDP-43 is predominantly nuclear but exits to the cytoplasm when it is not found in large assemblies. Increased TDP-43 levels in the cytoplasm is linked to pathology (see e.g., FIG. 31A). RNA-driven formation of large TDP-43 complexes in the nucleus helps retain TDP-43 in the nucleus, and overexpression of a specific RNA transcript increases TDP-43 nuclear retention (see e.g., FIG. 31B-FIG. 31D).

What is claimed is:

1. A method of preventing or reducing aggregation, misfolding, or intracellular seeding of TAR DNA-binding protein (TDP-43) in a subject in need thereof comprising administering a therapeutically effective amount of a synthetic oligonucleotide having TDP-43 binding activity.

2. The method of claim 1, wherein the synthetic oligonucleotide comprises at least about ten nucleotides and is at least 50% identical to SEQ ID NO: 1, SEQ ID: NO: 2, SEQ ID NO: 3, SEQ ID NO: 4, or SEQ ID NO: 5 or is at least 50% identical to a corresponding, reverse, complement, or reverse-complement nucleotide sequence of SEQ ID NO: 1, SEQ ID NO: 2, SEQ ID NO: 3, SEQ ID NO: 4, or SEQ ID NO: 5.

3. The method of claim 1, wherein the synthetic oligonucleotide comprises SEQ ID NO: 1, SEQ ID: NO: 2, SEQ ID NO: 3, SEQ ID NO: 4, or SEQ ID NO: 5 or comprises a corresponding, reverse, complement, or reverse-complement nucleotide sequence of SEQ ID NO: 1, SEQ ID NO: 2, SEQ ID NO: 3, SEQ ID NO: 4, or SEQ ID NO: 5.

4. The method of claim 1, wherein the synthetic oligonucleotide comprises at least about 6, about 12, about 18, or about 30 (GU) repeats.

5. The method of claim 1, wherein the synthetic oligonucleotide comprises at least two TDP-43 binding sites.

6. The method of claim 1, wherein the subject has or is suspected of having a neurodegenerative disease, disorder, or condition.

7. The method of claim 6, wherein the neurodegenerative disease, disorder, or condition is Amyotrophic Lateral Sclerosis (ALS), frontotemporal dementia (FTD or FTLD-TDP-43), limbic-predominant age-related TDP-43 encephalopathy (LATE), Alzheimer's disease, multisystem proteinopathy, or chronic traumatic encephalopathy.

8. The method of claim 1, wherein the synthetic oligonucleotide is a modified synthetic oligonucleotide comprising one or more backbone, sugar moiety, or nucleic base modifications.

9. The method of claim 8, wherein the modified synthetic oligonucleotide comprises a 2′-O-methyl base, 2′-O-methoxyethyl base, or phosphorothioate bond modification.

10. The method of claim 8, wherein the modified synthetic oligonucleotide comprises at least about ten nucleotides and is at least 50% identical to SEQ ID NO: 6, SEQ ID: NO: 7, SEQ ID NO: 8, or SEQ ID NO: 9, or is at least 50% identical to a corresponding, reverse, complement, or reverse-complement nucleotide sequence of SEQ ID NO: 6, SEQ ID NO: 7, SEQ ID NO: 8, or SEQ ID NO: 9.

11. The method of claim 8, wherein the modified synthetic oligonucleotide comprises SEQ ID NO: 6, SEQ ID: NO: 7, SEQ ID NO: 8, or SEQ ID NO: 9, or comprises a corresponding, reverse, complement, or reverse-complement nucleotide sequence of SEQ ID NO: 6, SEQ ID NO: 7, SEQ ID NO: 8, or SEQ ID NO: 9.

12. A pharmaceutical composition comprising an isolated oligonucleotide having TDP-43 binding activity and at least 80% identity to a sequence selected from one of SEQ ID NO: 1, SEQ ID NO: 2, SEQ ID NO: 3, SEQ ID NO: 4, or SEQ ID NO: 5.

13. A method of treating a neurodegenerative disease, disorder, or condition in a subject in need thereof comprising administering a therapeutically effective amount of a synthetic oligonucleotide having TDP-43 binding activity.

14. The method of claim 13, wherein the synthetic oligonucleotide comprises at least about ten nucleotides and is at least 50% identical to SEQ ID NO: 1, SEQ ID: NO: 2, SEQ ID NO: 3, SEQ ID NO: 4, or SEQ ID NO: 5 or is at least 50% identical to a corresponding, reverse, complement, or reverse-complement nucleotide sequence of SEQ ID NO: 1, SEQ ID NO: 2, SEQ ID NO: 3, SEQ ID NO: 4, or SEQ ID NO: 5.

15. The method of claim 13, wherein the synthetic oligonucleotide comprises at least about 6, about 12, about 18, or about 30 (GU) repeats.

16. The method of claim 13, wherein the neurodegenerative disease, disorder, or condition is Amyotrophic Lateral Sclerosis (ALS), frontotemporal dementia (FTD or FTLD-TDP-43), limbic-predominant age-related TDP-43 encephalopathy (LATE), Alzheimer's disease, multisystem proteinopathy, or chronic traumatic encephalopathy.

17. The method of claim 13, wherein the synthetic oligonucleotide is a modified synthetic oligonucleotide comprising one or more backbone, sugar moiety, or nucleic base modifications.

18. The method of claim 17, wherein the modified synthetic oligonucleotide comprises a 2′-O-methyl base, 2′-O-methoxyethyl base, or phosphorothioate bond modification.

19. The method of claim 17, wherein the modified synthetic oligonucleotide comprises at least about ten nucleotides and is at least 50% identical to SEQ ID NO: 6, SEQ ID: NO: 7, SEQ ID NO: 8, or SEQ ID NO: 9, or is at least 50% identical to a corresponding, reverse, complement, or reverse-complement nucleotide sequence of SEQ ID NO: 6, SEQ ID NO: 7, SEQ ID NO: 8, or SEQ ID NO: 9.

20. The method of claim 17, wherein the modified synthetic oligonucleotide comprises SEQ ID NO: 6, SEQ ID: NO: 7, SEQ ID NO: 8, or SEQ ID NO: 9, or comprises a corresponding, reverse, complement, or reverse-complement nucleotide sequence of SEQ ID NO: 6, SEQ ID NO: 7, SEQ ID NO: 8, or SEQ ID NO: 9.
